(19) United States
(12) Patent Application Publication
(10) Pub. No.: US 2025/0032613 A1
(43) Pub. Date: Jan. 30, 2025

(54) Systems and Methods for Treating Graves' Disease

(71) Applicant: Brigham Young University, Provo, UT (US)

(72) Inventors: Scott Weber, Lindon, UT (US); Abigail Cheever, Provo, UT (US); Kim O'Neill, Provo, UT (US)

(73) Assignee: Brigham Young University, Provo, UT (US)

(21) Appl. No.: 18/745,948

(22) Filed: Jun. 17, 2024

Related U.S. Application Data
(60) Provisional application No. 63/521,269, filed on Jun. 15, 2023.

(51) Int. Cl. A61K 39/00; C12N 5/0783

(52) U.S. Cl. CPC A61K 39/4611; A61K 39/4631; A61K 39/46433; C12N 5/0636; A61K 2239/21; C12N 2510/00

(57) ABSTRACT

A novel treatment for Graves' Disease (GD) is disclosed and described. Chimeric autoantigen receptor (CAAR) T cells are engineered using a CAAR construct causing thyroid stimulating hormone receptor (TSHR) epitope expression such that the engineered CAAR T cells serve as bait for autoreactive B cells. The engineered CAAR T cells specifically eliminate the autoreactive B cells, thus eliminating the causative factor for GD. Certain CAAR T cells are further engineered to incorporate bispecific LINK CAR technology to further require the presence of CD19 or BCMA to further increase CAAR T cell targeting specificity to autoreactive B cells and plasma cells.

### CROSS-REFERENCE TO RELATED APPLICATIONS

This application claims the benefit of U.S. Provisional Patent Application No. 63/521,269, titled Characterization of a Novel Graves' Disease Treatment that Uses Chimeric Autoantigen Receptor Technology and filed Jun. 15, 2023, which is incorporated herein by reference for all it discloses.

### BACKGROUND OF THE INVENTION

### 1. Field of the Invention

The present invention relates to treatment of disease, and more particularly to methods for permanent treatment of Graves' Disease.

### 2. Background and Related Art

Graves' disease (GD) is an antibody-mediated autoimmune disease of the thyroid gland. It is one of the most prevalent autoimmune diseases, and it most frequently affects women between 30-50 years of age [1]. Symptoms of GD are those typical of hyperthyroidism, with high levels of thyroid hormones affecting metabolism and growth; however, symptoms in other organs can occur as well [2]. A common complication is Graves' ophthalmopathy, which causes severe inflammation around the eyes and affects about 25-30% of GD patients [3].

GD pathogenesis centers on autoreactive B cells that produce antibodies (Abs) which bind the thyroid-stimulating hormone receptor (TSHR) and stimulate the thyroid, causing hyperthyroidism. These anti-TSHR antibodies (TRAbs) are considered the cause of disease in GD patients. These autoreactive B cells and their production of TRAbs are induced through helper T cell stimulation (see FIG. 1, which illustrates how autoreactive T cells provide T cell help to autoreactive B cells, which then produce Abs that bind to TSHR; TRAb binding to TSHR causes hormone production and hyperthyroidism) [4]. As with many autoimmune diseases, however, the root cause of GD remains unknown. It is suspected that many factors play a role, including genetics and epigenetics, the gut microbiome, and the imbalance of immune cell development and activation [5]. Patients with GD are also at significantly higher risk of developing thyroid cancers, and GD-associated thyroid cancer is more aggressive [6-8].

There are three categories of TRAbs (TSHR-stimulating (agonist) Abs, TSHR-blocking (antagonist) Abs, and neutral Abs) and all three of these types of TSHR Abs can be found in GD patients [9,10]. TSHR-stimulating Abs are found in all GD patients, and they bind to the leucine rich repeat (LRR) region of TSHR, the region where TSH binds TSHR as well (see FIG. 2, which illustrates TRAb classes and their binding epitopes) [11]. TSHR-blocking Abs also bind to the LRR, and can block TSH binding, and TSHR-neutral Abs bind to other regions besides the LRR (e.g., the hinge) and don't have significant physiological effect. Stimulating TRAbs lead to the classical pathogenesis of GD by mimicking the activity of TSH in stimulating production of thyroid hormones. Binding to the LRR is what stimulates the chronic activation and production of thyroid hormones. The conformation of this region is necessary for both binding to occur and for the conformational change of TSHR [12]. Stimulating TRAbs are typically IgG class 2 molecules [10].

The main treatments for GD are antithyroid drugs (ATDs), radioactive iodine therapy (RAI), and in some cases, thyroidectomy [13]. ATDs are generally the first-line treatment, including methimazole, propylthiouracil, and carbimazole. These drugs inhibit the enzyme thyroid peroxidase, leading to a decrease in the production of thyroid hormones. The majority of patients relapse into hyperthyroidism after stopping this treatment [3]. RAI is usually used after ATDs fail. This treatment attacks the thyroid tissue, slowly reducing it over time. RAI leads to hypothyroidism, necessitating hormone replacement therapy. For severe cases, thyroidectomy is a fast-acting option, though it also necessitates lifelong hormone replacement therapy post-surgery [3]. There currently are not any treatments available to patients which address the disease-causing element of GD.

CAR T cell components and mechanism: CAR T cells are a relatively new immunotherapy that harness the cytotoxic function of T cells in a targeted and more-potent manner. This therapy uses synthetic biology to target engineered T cells towards a certain antigen, without requiring major histocompatibility complex (MHC) binding or costimulation from receptors or cytokines.

CAR constructs contain several different domains that enable T cells to bind with a specific antigen and activate cytotoxicity with a single step [14]. The binding region is commonly a single chain variable fragment (scFv), which is an engineered protein based on the variable regions of the heavy and light chains of Abs (see FIG. 3, which shows that CAR constructs contain an scFv that binds to an antigen, a transmembrane domain, and activation domains (CD28 or F-1BB and CD3ζ; perforin/granzymes are directionally released upon activation) [15]. The scFv is connected to a transmembrane domain by a hinge region, which then connects to signaling domains. These signaling domains allow the CAR T cell to activate cytotoxicity in one step. First generation CARs only contained the primary signaling domain CD3ζ, while later generations improved efficacy by adding additional signaling domains, commonly including CD28 or 4-1BB, though others are being introduced [14,16]. CAR T cell therapy is primarily ex vivo, where a patient's T cells are isolated, genetically modified with the CAR, expanded and purified, and returned to the patient intravenously [17].

History and current successes of CAR T cell therapy: CARs were first conceptualized as a one step, antigen-specific activation of T cells in 1987 by combining aspects of B and T cell receptors [18]. In 1989, CAR T cells were proposed as a possible immunotherapy; however, these preliminary CAR T cells contained only CD3ζ and were not functional without costimulation [19]. The first effective CAR T cell was created in 1998 by adding the costimulatory domain CD28 to the CAR [20]. These developments were then applied to create CAR T cells that eliminated leukemia cells, first in vitro and then in a mouse model [21,22]. Next-generation CAR T cells have been developed to fix persistence problems and have been used as viable treatments for hematological cancers. In 2010 the first Phase I clinical trials for CAR T cell therapy began, leading to FDA approval in 2019 [23]. Recently, the first CAR T cell patients were shown to have CAR T cells still circulating in their bloodstream 10 years after the treatment was administered and were still in complete remission, demonstrating the persistence and success of CAR T cell therapy [24].

CAR T-cell therapies were first FDA approved for treatment of pediatric and young adult acute lymphoblastic leukemia (ALL) and later other B cell malignancies. Tisagenlecleucel (Kymriah™), developed by Novartis, was the first to be approved, with Axicabtagene ciloleucel (Yescarta™) developed by Kite Pharma following several months later [25]. CAR T cell research rapidly expanded following these successes, and currently there are six FDA approved CAR T-cell therapies, all for hematological cancers (Abecma™, Breyanzi™, Carvykti™, Kymriah™, Tecartus™, and Yescarta™) [15,26,27]. The overall response rate for patients treated across all FDA approved CAR T cells therapies is over 80% [27]. CAR T cell therapy is quickly becoming a more common treatment for patients with relapsed and refractory hematological cancers [17]. CAR T cell therapy has had its greatest successes thus far in B cell cancers. Five out of six of the current FDA approved CAR T cell therapies target a surface marker called CD19 found on all B cells [15]. This treatment eliminates all B cells, leaving the patient partially immunosuppressed, though their B cell cancer is often completely eradicated [26,28].

CAR T cells have powerful cytotoxic capabilities, making a high level of specificity important for their safety. One way to improve the specificity of CAR T cells is creating a bispecific CAR that requires binding to two antigens before activation can occur [29]. This reduces the potential of on-target, off-tumor activation. Researchers have been attempting to develop such a bispecific CAR, but it has proven difficult to make completely bispecific CARs that are effective in the standard format. SPLIT CAR T cells separate the CD3 primary signaling domain and the CD28 or 4-1BB costimulatory domain into two separate CAR constructs, with separate scFvs (see FIG. 4 at A) [30]. Full activation of this CAR is only achieved upon binding of both antigens; however, binding to the CD3 CAR alone still causes a weaker activation that can induce some cytotoxic effects [30]. SynNotch CAR T cells use a cleaved transcription factor; they are primed by binding of the first antigen to a synthetic notch CAR which releases a transcription factor that enables transcription of a traditional CAR (see FIG. 4 at B) [31]. Then a second antigen can activate the CAR after it is expressed and surface localized [30]. This system improves the bispecificity, but on-target, off-tumor activation can still occur because the effect of the transcription factor can last long enough for the two binding events to occur with two different cells [32]. LINK CAR T cells have separate and novel downstream activation molecules; they have two CAR constructs that each have a T cell downstream signaling molecule as the activation domain (see FIG. 4 at C) [33]. When a few key mutations are made to SLP-76 and LAT as the activation domains, they form a true bispecific CAR where

both antigens are required to bind for activation of the CAR T cell [33]. Data has also shown that LINK CARs also present lower levels of exhaustion markers after consistent stimulation than traditional CAR T cells. Thus, LINK CARs present a promising option for improving the specificity of CAR T cells in various applications [33].

CAR T cell research is still growing, with over 500 clinical trials currently underway [15]. Much of this research is focused on making CAR T cells viable for treatment of solid tumor cancers; however, there are many roadblocks which still need to be overcome on this front [34]. A much smaller subset of CAR T cell research is focused on applying this immunotherapy method to other diseases, primarily to autoimmune disease.

CAR T cell therapies for autoimmune disease: Autoantibody mediated autoimmune

diseases have recently become a target for CAR T cell application. Elimination of all B cells or elimination of just autoreactive B cells by CAR T cells would stop the production of autoantibodies and should relieve the patient from the disease. The efficiency of CAR T cells at eliminating B cells, as shown in hematological cancers, makes this a logical treatment option.

Repurposing CAR T cells for B cell autoimmune diseases: B cell-depleting CAR T cells have been repurposed as a treatment for B cell-mediated autoimmune diseases, first for systemic lupus erythematosus (SLE). Several B cell depletion drugs and monoclonal Abs have been used to eliminate B cell populations in SLE patients, but they have only been partially effective at eliminating B cells [35-37]. Anti-CD19 CAR T cells were repurposed for B cell depletion in SLE patients, as shown by completely depleted B cell populations in two SLE mouse models [38]. Ab levels were also depleted, and clinical manifestation of SLE in the mice was greatly improved as well [39]. In a Phase I clinical trial anti-CD19 CAR T cells were safe and well tolerated, and after a median of three months following treatment, all patients achieved full SLE remission [40]. The patients also showed return of naïve B cell populations about 100 days after treatment, allowing patients to reform B cell protective immunity, though patients have not shown a return of autoreactive B cell populations as far as currently seen [40].

Recently a patient with severe multidrug-resistant dermatomyositis (anti-synthetase syndrome) was also treated with anti-CD19 CAR T cells and achieved similar results to the SLE patients treated prior [41]. Further clinical trials are underway using anti-CD19 CAR T cells for treatment of several B cell-mediated autoimmune disorders, including SLE (NCT06121297), idiopathic inflammatory myopathy (NCT06154252), systemic sclerosis (NCT06328777), and myasthenia gravis (NCT06359041).

Repurposed CAR T cells are beginning to show promise in treating autoimmune diseases as shown by the above trials. However, CD19 targeting CAR T cells eliminate all B cells. Though B cell populations can recover after time, some previous immunity can be lost, and there is a period when patients are significantly immunosuppressed. This may still be a viable option for people suffering with severe B cell mediated autoimmune diseases, as with SLE. Though, the resulting immunosuppression makes this a less appealing treatment. It also may not be justifiable for less severe autoimmune diseases.

Chimeric autoantigen receptor (CAAR) T cells for autoantibody mediated autoimmune diseases: Chimeric autoantigen receptor (CAAR) T cells are a novel development of CAR T cells which are designed to specifically eliminate autoreactive B cells, rather than eliminating all B cells (see FIG. 5). This is accomplished by replacing the scFv as the binding domain with a piece of an autoantigen that will act as bait for the autoreactive B cells, which will bind via their B cell receptors (BCRs) [42]. This method is dependent on the fact that B cells produce both Abs and B cell receptors (BCRs) specific for the same antigens, thus the autoantigen that the

autoantibodies bind to will also be bound by the BCRs of the B cells producing them [43]. CAAR T cells were pioneered for treatment of pemphigus vulgaris (PV), a skin autoimmune disease, where autoantibodies against Dsg3, a keratinocyte adhesion protein, cause blistering and inflammation [44,45]. A panel of CAAR T cells with epitopes of DSG3 were engineered, and they showed successful cytotoxic effects against an engineered DSG3 B cell line and patient-derived DSG3 B cells [44]. Consideration was taken as to whether soluble anti-Dsg3 Abs would inhibit the CAAR T cell function or initiate anergy; however, in the presence of PV serum, the soluble Abs did not have an inhibitory effect on the CAAR cytotoxicity and may even have a beneficial effect in preventing exhaustion [44]. In a mouse model the same findings were replicated, and the mice showed reduction of blistering, notably with no off-target effects recorded on healthy B cells [44]. Preliminary data from PV patient blood samples quantifying dosage pharmacology were recently published, qualifying it for Phase I clinical trials (NCT04422912), which are currently ongoing [46]. This team also recently applied the same principle to treatment of muscle-specific tyrosine kinase myasthenia gravis (MuSK MG) for MuSK specific B cell depletion [47]. These MuSK CAAR T cells effectively reduced MuSK B cell and Ab populations without affecting healthy B cells or normal Ab levels, and it has also recently begun a Phase 1 clinical trial (NCT05451212) [47].

One potential drawback to CAAR T cells comes from the fact that the binding domain is an epitope of a protein occurring naturally in the body. These proteins may have natural binding partners which have the potential to activate the CAAR T cell, causing off-target effects. The soluble Abs produced by the autoreactive B cells also inherently bind to the CAAR T cells, and it is unclear how they will affect the CAAR T cell's function in every circumstance [44].

### BRIEF SUMMARY OF THE INVENTION

A novel treatment for Graves' Disease (GD) is disclosed and described. Chimeric autoantigen receptor (CAAR) T cells are engineered using a CAAR construct causing thyroid stimulating hormone receptor (TSHR) epitope expression such that the engineered CAAR T cells serve as bait for autoreactive B cells. The engineered CAAR T cells specifically eliminate the autoreactive B cells, thus eliminating the causative factor for GD. Certain CAAR T cells are further engineered to incorporate bispecific LINK CAR technology to further require the presence of CD19 or BCMA to further increase CAAR T cell targeting specificity to autoreactive B cells and plasma cells.

There currently are not any treatments available to patients which address the disease-causing element of GD. A treatment mechanism that could specifically and permanently eliminate the autoreactive B cells and their subsequent TRAbs could provide a cure for patients with GD. Such a treatment is provided by engineered CAAR T cells expressing TSHR epitopes to serve as bait for autoreactive B cells. When autoreactive B cells bind to the TSHR epitopes, the CAAR T cells are activated, causing directional cytotoxic effects. A bispecific LINK CAR that requires binding of an autoantigen as well as a B cell specific marker could eliminate any possible off target activation from soluble Abs as well as other proteins that can interact with the CAAR binding domain. The bispecific LINK CAR addresses the potential drawback to CAAR T cells from the fact that the binding domain is an epitope of a protein occurring naturally in the body, limiting off-target effects.

CAAR T cells are novel in their specific targeting of autoreactive B cells, overcoming the disadvantages of complete B cell elimination and subsequent immunosuppression [48]. This CAAR T cell treatment method could be critical to the treatment of autoantibody mediated autoimmune diseases in the future. Additionally, the application of LINK CAR technology provides additional specificity to CAAR T cells, eliminating activation by soluble Abs and other proteins that could interact with the autoantigen binding domain. This addition to CAAR T cells makes them a safer and more promising treatment option for patients suffering from autoantibody mediated autoimmune diseases.

According to implementations of the invention, a method for treating Graves' Disease (GD) includes engineering and creating a chimeric autoantigen receptor (CAAR) T cell line for a patient including. Engineering and creating the CAAR T cell line includes selecting a thyroid stimulating hormone receptor (TSHR) epitope predicted to fold autonomously and to bind effectively to a disease-causing, stimulating anti-TSHR antibody (TRAb) of a B cell, synthesizing a DNA fragment for the TSHR epitope, cloning the DNA fragment into a CAAR construct as a binding domain, the CAAR construct including a vector containing an extracellular binding domain and linker, a transmembrane domain, and a signaling domain, transfecting the CAAR construct into a lentiviral-generating cell, collecting and isolating lentivirus for the CAAR construct, and transducing T cells of the patient with the lentivirus to generate the CARR T cell line. The method further includes confirming expression of the CARR construct in the CARR T cell line, and administering a therapeutic dose of the CARR T cell line expressing the CARR construct to the patient.

In some implementations, the TSHR epitope includes a leucine rich repeat (LRR) region where disease-causing, stimulating TRAbs bind. In some implementations, the transmembrane domain includes a CD28 transmembrane domain, and the signaling domain includes CD28 and CD3ζ signaling domains. In some implementations, transfecting the CARR construct into a lentiviral-generating cell includes co-transfecting the lentiviral-generating cell with the CARR construct and lentiviral packaging and envelope plasmids. In some implementations, the lentiviral-generating cell that is transfected is a HEK293FT cell.

In some implementations, administering the therapeutic dose of the CARR T cell line expressing the CARR construct causes destruction of substantially all autoreactive B cells of the patient expressing TRAbs while not affecting substantially any other B cells of the patient. In some implementations, engineering and creating a CAAR T cell line further includes incorporating a LINK CARR construct in the CAAR T cell line that requires binding of CD19 before the CAAR T cell line is activated, enhancing specificity of the CAAR T cell line to B cells. In some implementations, the TSHR epitope includes an amino acid size of 260 to 290 amino acids (aa). In some implementations, the TSHR epitope includes an amino acid size of 270 to 280aa. In some implementations, the TSHR epitope includes an amino acid size of 270 to 290aa.

According to some implementations of the invention, a method for treating an autoimmune disease includes engineering and creating a chimeric autoantigen receptor (CAAR) T cell line for a patient. The step for engineering and creating a CAAR T cell line includes steps of selecting a receptor epitope predicted to fold autonomously and to bind effectively to a disease-causing antibody (Ab) of a cell of the patient, synthesizing a DNA fragment for the receptor epitope, cloning the DNA fragment into a CAAR construct as a binding domain, the CAAR construct including a vector containing an extracellular binding domain and linker, a transmembrane domain, and a signaling domain, transfecting the CAAR construct into a lentiviral-generating cell, collecting and isolating lentivirus for the CAAR construct, and transducing T cells of the patient with the lentivirus to generate the CARR T cell line. The method further includes confirming expression of the CARR construct in the CARR T cell line, and administering a therapeutic dose of the CARR T cell line expressing the CARR construct to the patient.

In some implementations, the epitope includes a leucine rich repeat (LRR) region where disease-causing, stimulating Abs bind. In some implementations, the transmembrane domain includes a CD28 transmembrane domain, and the signaling domain includes CD28 and CD3ζ signaling domains. In some implementations, transfecting the CARR construct into a lentiviral-generating cell includes co-transfecting the lentiviral-generating cell with the CARR construct and lentiviral packaging and envelope plasmids. In some implementations, the lentiviral-generating cell that is transfected is a HEK293FT cell.

In some implementations, administering the therapeutic dose of the CARR T cell line expressing the CARR construct causes destruction of substantially all autoreactive disease-causing cells of the patient expressing Abs while not affecting substantially any other non-disease-causing cells of the patient. In some implementations, engineering and creating a CAAR T cell line further includes incorporating a LINK CARR construct in the CAAR T cell line that requires binding of a cell-type-specific receptor before the CAAR T cell line is activated, enhancing specificity of the CAAR T cell line to disease-causing cells. In some implementations, the epitope includes an amino acid size of 260 to 290aa. In some implementations, the epitope includes an amino acid size of 270 to 280aa. In some implementations, the epitope includes an amino acid size of 270 to 290aa.

According to further implementations of the invention, a method for treating Graves' Disease (GD) includes engineering and creating a chimeric autoantigen receptor (CAAR) T cell line for a patient. The step of engineering and creating a CAAR T cell line includes selecting a thyroid stimulating hormone receptor (TSHR) epitope including a leucine rich repeat (LRR) region predicted to fold autonomously and to bind effectively to a disease-causing, stimulating anti-TSHR antibody (TRAb) of a B cell, synthesizing a DNA fragment for the TSHR epitope, the DNA fragment including: a TSHR epitope sequence, a GS linker after the TSHR epitope sequence, and a BpiI restriction site at each end to facilitate cloning, cloning the DNA fragment into a CAAR construct as a binding domain, the CAAR construct including a vector containing an extracellular binding domain and linker, a CD28 transmembrane domain, and CD28 and CD3ζ signaling domains, co-transfecting the CAAR construct into a lentiviral-generating cell with lentiviral packaging and envelope plasmids, collecting and isolating lentivirus for the CAAR construct, and transducing T cells of the patient with the lentivirus to generate the CARR T cell line. The method further includes administering a therapeutic dose of the CARR T cell line expressing the CARR construct to the patient.

In some implementations, the method further includes confirming expression of the CARR construct in the CARR T cell line. In some implementations, engineering and creating a CAAR T cell line further includes incorporating a LINK CARR construct in the CAAR T cell line that requires binding of CD19 before the CAAR T cell line is activated, enhancing specificity of the CAAR T cell line to B cells. In some implementations, the TSHR epitope includes an amino acid size of 270 to 290aa. In some implementations, the TSHR epitope includes an amino acid size of 260 to 290aa. In some implementations, the TSHR epitope includes an amino acid size of 270 to 280aa.

### BRIEF DESCRIPTION OF THE SEVERAL VIEWS OF THE DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a diagrammatic view of the immunopathogenesis of Graves' Disease;

FIG. 2 shows a diagrammatic view of anti-thyroid-stimulating hormone (TSHR) antibodies (Abs) (TRAbs)

FIG. 3 shows a diagrammatic view of a chimeric antigen receptor (CAR) T cell adjacent a cancer cell;

FIG. 4 shows a diagrammatic view of various bispecific CAR structures;

FIG. 5 shows a diagrammatic view of a chimeric autoantigen receptor (CAAR) T cell adjacent an autoreactive B cell;

FIG. 6 shows a protein model of a thyroid stimulating hormone receptor (TSHR) with a gene composition and epitope selection;

FIG. 7 illustrates an exemplary CAAR construct;

FIG. 8 shows flow cytometry binding data for B-lymphocyte antigen CD19 (CD19) and various CAAR constructs (CAAR1, CAAR2, and CAAR2.7);

FIG. 9 shows flow cytometry CD69 activation data;

FIG. 10 provides flow cytometry data showing transduction efficiencies of different CAR lentivirus into primary human T cells;

FIG. 11 shows simplified plasmid maps of engineered anti-TSHR B cell lines;

FIG. 12 shows measured data of B cell receptors (BCR) expression in engineered anti-TSHR B cell lines;

FIG. 13 shows preliminary flow cytometry cytotoxicity data;

FIG. 14 shows an illustrative Link CAAR constructs map; and

FIG. 15 shows expression and binding of LINK CAR T cells in Jurkat cells to their specific antigen.

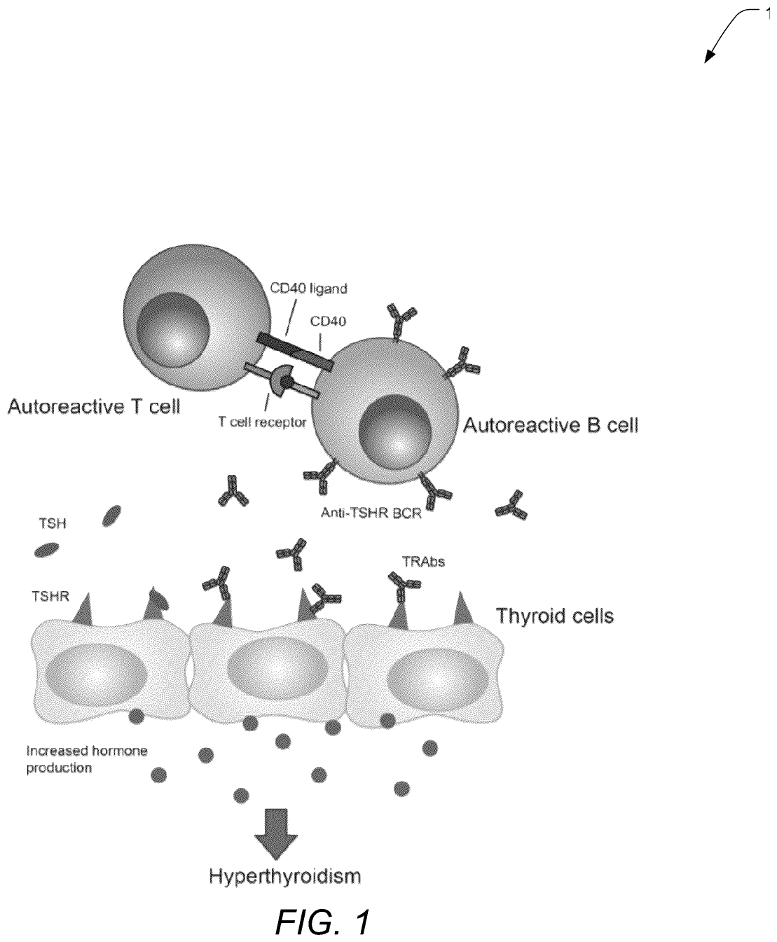

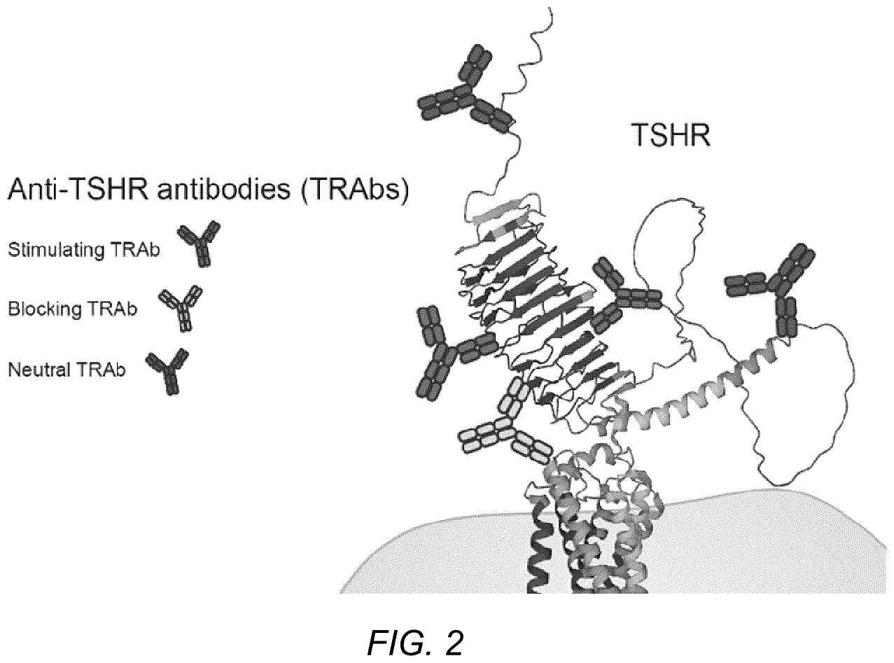

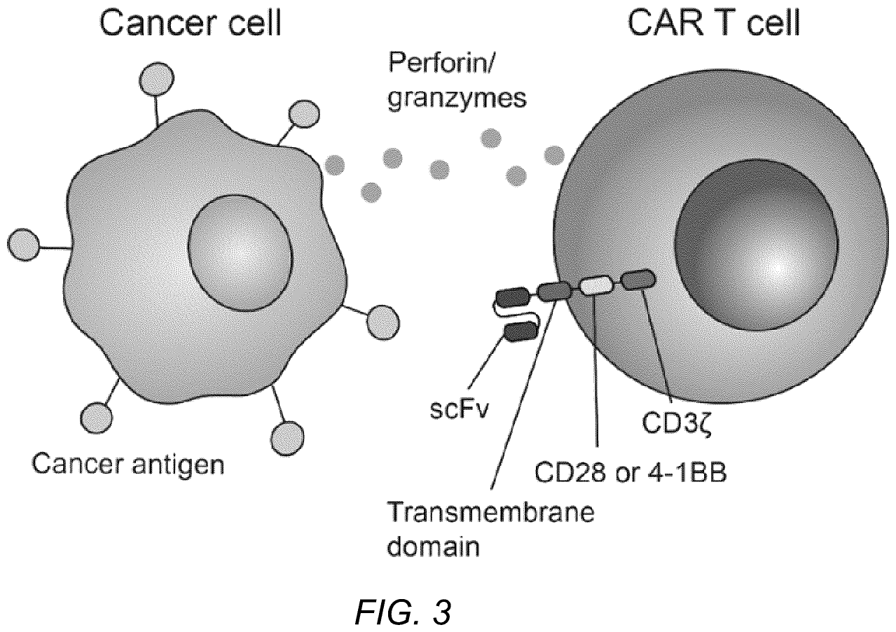

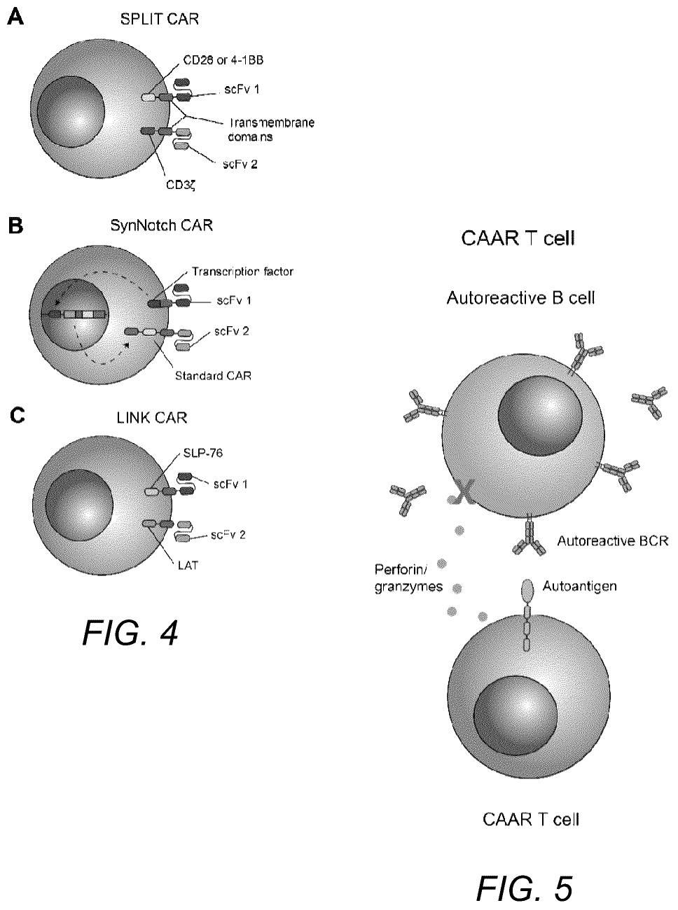

### DETAILED DESCRIPTION OF THE INVENTION

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

A novel treatment for Graves' Disease (GD) is disclosed and described. Chimeric autoantigen receptor (CAAR) T cells are engineered using a CAAR construct causing thyroid stimulating hormone receptor (TSHR) epitope expression such that the engineered CAAR T cells serve as bait for autoreactive B cells. The engineered CAAR T cells specifically eliminate the autoreactive B cells, thus eliminating the causative factor for GD. Certain CAAR T cells are further engineered to incorporate bispecific LINK CAR technology to further require the presence of CD19 or BCMA to further increase CAAR T cell targeting specificity to autoreactive B cells and plasma cells.

There currently are not any treatments available to patients which address the disease-causing element of GD. A treatment mechanism that could specifically and permanently eliminate the autoreactive B cells and their subsequent TRAbs could provide a cure for patients with GD. Such a treatment is provided by engineered CAAR T cells expressing TSHR epitopes to serve as bait for autoreactive B cells. When autoreactive B cells bind to the TSHR epitopes, the CAAR T cells are activated, causing directional cytotoxic effects. A bispecific LINK CAR that requires binding of an autoantigen as well as a B cell specific marker could eliminate any possible off target activation from soluble Abs as well as other proteins that can interact with the CAAR binding domain. The bispecific LINK CAR addresses the potential drawback to CAAR T cells from the fact that the binding domain is an epitope of a protein occurring naturally in the body, limiting off-target effects.

According to embodiments of the invention, a method for treating Graves' Disease (GD) includes engineering and creating a chimeric autoantigen receptor (CAAR) T cell line for a patient including. Engineering and creating the CAAR T cell line includes selecting a thyroid stimulating hormone receptor (TSHR) epitope predicted to fold autonomously and to bind effectively to a disease-causing, stimulating anti-TSHR antibody (TRAb) of a B cell, synthesizing a DNA fragment for the TSHR epitope, cloning the DNA fragment into a CAAR construct as a binding domain, the CAAR construct including a vector containing an extracellular binding domain and linker, a transmembrane domain, and a signaling domain, transfecting the CAAR construct into a lentiviral-generating cell, collecting and isolating lentivirus for the CAAR construct, and transducing T cells of the patient with the lentivirus to generate the CARR T cell line. The method further includes confirming expression of the CARR construct in the CARR T cell line, and administering a therapeutic dose of the CARR T cell line expressing the CARR construct to the patient.

In some embodiments, the TSHR epitope includes a leucine rich repeat (LRR) region where disease-causing, stimulating TRAbs bind. In some embodiments, the transmembrane domain includes a CD28 transmembrane domain, and the signaling domain includes CD28 and CD3ζ signaling domains. In some embodiments, transfecting the CARR construct into a lentiviral-generating cell includes co-transfecting the lentiviral-generating cell with the CARR construct and lentiviral packaging and envelope plasmids. In some embodiments, the lentiviral-generating cell that is transfected is a HEK293FT cell.

In some embodiments, administering the therapeutic dose of the CARR T cell line expressing the CARR construct causes destruction of substantially all autoreactive B cells of the patient expressing TRAbs while not affecting substantially any other B cells of the patient. In some embodiments, engineering and creating a CAAR T cell line further includes incorporating a LINK CARR construct in the CAAR T cell line that requires binding of CD19 before the CAAR T cell line is activated, enhancing specificity of the CAAR T cell line to B cells. In some embodiments, the TSHR epitope includes an amino acid size of 260 to 290 amino acids (aa). In some embodiments, the TSHR epitope includes an amino acid size of 270 to 280aa. In some embodiments, the TSHR epitope includes an amino acid size of 270 to 290aa.

According to some embodiments of the invention, a method for treating an autoimmune disease includes engineering and creating a chimeric autoantigen receptor (CAAR) T cell line for a patient. The step for engineering and creating a CAAR T cell line includes steps of selecting a receptor epitope predicted to fold autonomously and to bind effectively to a disease-causing antibody (Ab) of a cell of the patient, synthesizing a DNA fragment for the receptor epitope, cloning the DNA fragment into a CAAR construct as a binding domain, the CAAR construct including a vector containing an extracellular binding domain and linker, a transmembrane domain, and a signaling domain, transfecting the CAAR construct into a lentiviral-generating cell, collecting and isolating lentivirus for the CAAR construct, and transducing T cells of the patient with the lentivirus to generate the CARR T cell line. The method further includes confirming expression of the CARR construct in the CARR T cell line, and administering a therapeutic dose of the CARR T cell line expressing the CARR construct to the patient.

In some embodiments, the epitope includes a leucine rich repeat (LRR) region where disease-causing, stimulating Abs bind. In some embodiments, the transmembrane domain includes a CD28 transmembrane domain, and the signaling domain includes CD28 and CD3ζ signaling domains. In some embodiments, transfecting the CARR construct into a lentiviral-generating cell includes co-transfecting the lentiviral-generating cell with the CARR construct and lentiviral packaging and envelope plasmids. In some embodiments, the lentiviral-generating cell that is transfected is a HEK293FT cell.

In some embodiments, administering the therapeutic dose of the CARR T cell line expressing the CARR construct causes destruction of substantially all autoreactive disease-causing cells of the patient expressing Abs while not affecting substantially any other non-disease-causing cells of the patient. In some embodiments, engineering and creating a CAAR T cell line further includes incorporating a LINK CARR construct in the CAAR T cell line that requires binding of a cell-type-specific receptor before the CAAR T cell line is activated, enhancing specificity of the CAAR T cell line to disease-causing cells. In some embodiments, the epitope includes an amino acid size of 260 to 290aa. In some embodiments, the epitope includes an amino acid size of 270 to 280aa. In some embodiments, the epitope includes an amino acid size of 270 to 290aa.

According to further embodiments of the invention, a method for treating Graves' Disease (GD) includes engineering and creating a chimeric autoantigen receptor (CAAR) T cell line for a patient. The step of engineering and creating a CAAR T cell line includes selecting a thyroid stimulating hormone receptor (TSHR) epitope including a leucine rich repeat (LRR) region predicted to fold autonomously and to bind effectively to a disease-causing, stimulating anti-TSHR antibody (TRAb) of a B cell, synthesizing a DNA fragment for the TSHR epitope, the DNA fragment including: a TSHR epitope sequence, a GS linker after the TSHR epitope sequence, and a BpiI restriction site at each end to facilitate cloning, cloning the DNA fragment into a CAAR construct as a binding domain, the CAAR construct including a vector containing an extracellular binding domain and linker, a CD28 transmembrane domain, and CD28 and CD3ζ signaling domains, co-transfecting the CAAR construct into a lentiviral-generating cell with lentiviral packaging and envelope plasmids, collecting and isolating lentivirus for the CAAR construct, and transducing T cells of the patient with the lentivirus to generate the CARR T cell line. The method further includes administering a therapeutic dose of the CARR T cell line expressing the CARR construct to the patient.

In some embodiments, the method further includes confirming expression of the CARR construct in the CARR T cell line. In some embodiments, engineering and creating a CAAR T cell line further includes incorporating a LINK CARR construct in the CAAR T cell line that requires binding of CD19 before the CAAR T cell line is activated, enhancing specificity of the CAAR T cell line to B cells. In some embodiments, the TSHR epitope includes an amino acid size of 270 to 290aa. In some embodiments, the TSHR epitope includes an amino acid size of 260 to 290aa. In some embodiments, the TSHR epitope includes an amino acid size of 270 to 280aa.

CAAR T cells are novel in their specific targeting of autoreactive B cells, overcoming the disadvantages of complete B cell elimination and subsequent immunosuppression [48]. This CAAR T cell treatment method could be critical to the treatment of autoantibody mediated autoimmune diseases in the future. Additionally, the application of LINK CAR technology provides additional specificity to CAAR T cells, eliminating activation by soluble Abs and other proteins that could interact with the autoantigen binding domain. This addition to CAAR T cells makes them a safer and more promising treatment option for patients suffering from autoantibody mediated autoimmune diseases.

Promise for CAAR T cell therapy in GD: CAR T cells have transformed how hematological cancers are treated because of their highly specific and potent ability to eliminate cells. Following their success in hematological cancers, logically, they have been applied to elimination of B cells in autoimmune diseases. CD19-specific CAR T cells have been effective in reducing symptoms of SLE through total B cell elimination; however, the resulting immunosuppression is undesirable. CAAR T cells are expected to be a critical improvement upon this strategy by eliminating autoreactive B cells only. By presenting an epitope of an autoantigen as the extracellular binding domain of the CAAR, functioning as bait, the autoreactive B cells will bind and be eliminated by the CAAR T cell. GD is an ideal disease to be treated through CAAR T cell therapy because it is organ-specific and is mediated by TRAbs. CAAR T cell elimination of autoreactive, anti-TSHR B cells in GD would stop production of TRAbs and halt disease progression. The elimination of the B cells producing these GD autoreactive Abs should also help decrease the rates of Graves' ophthalmopathy.

The use of CAAR T cells to treat GD is innovative in its potential to provide a specific and curative treatment option to the estimated 2-3% of the population that suffers from GD. Lifelong treatment of this population places a significant burden on not only the patient, but also on their families, caregivers, and society at large [49]. Because CAR and CAAR T cells proliferate, form memory cells, and essentially become part of the patient's immune system, the CAAR T cell therapy for treatment of GD proposed here has the potential be a one-time, curative treatment. Currently, there are no treatments for GD that provide a similar permanent solution without serious side effects and lifetime hormone replacement therapy.

After work had begun on this potential GD treatment, a paper was published developing a CAAR T cell with a similar strategy [50]. They used TSHR as a binding domain to attract anti-TSHR B cells in Graves' Disease; however, there are some notable differences between the published work and the potential GD treatment. The published construct design is different in several components, including the epitope of TSHR chosen. They also evaluated the efficacy of their CAAR T cells only against an anti-TSHR mouse hybridoma. Their work lends credence to the goal of this project, as their CAAR T cells were able to specifically eliminate their anti-TSHR mouse hybridomas; however, more questions need to be answered to move this work forward.

Specifically, human B cells with patient-derived, anti-TSHR BCRs provide a more clinically relevant mechanism to study the efficacy of CAAR T cell efficacy. The published paper also does not take into consideration soluble Ab interaction, or the presence of other proteins which could interact with the CAAR T cell. A full GD solution will evaluate these interactions and their effect on this possible treatment strategy. This is necessary for the safety and efficacy of CAAR T cells if they are ever to be a treatment option for GD. The treatment modality discussed herein also pioneers the use of bispecific LINK CAARs for improved specificity.

Noting the possibility for off-target activation of CAAR T cells with other naturally interacting proteins and soluble Abs, the application of LINK CAR technology could be beneficial in increasing the specificity and safety of CAAR T cells. Requiring binding to TSHR and a B cell marker like CD19 improves the safety and quality of treatment for GD patients by preventing off-target activation. Described herein is the novel construction of bispecific LINK CAAR T cells, evaluation of their efficacy at eliminating anti-TSHR B cells, and comparison of their safety and specificity compared with original (non bispecific LINK) CAAR T cells. In the future, bispecific LINK CAAR T cells could also make other CAAR treatments for autoimmune disease safer and cause fewer or less-severe side effects for patients.

CAAR T cell therapy has the potential to treat many autoantibody-mediated autoimmune diseases in addition to GD. Autoimmune diseases including lupus, rheumatoid arthritis, and many others are increasing in prevalence in the United States; however, treatment innovation has not yet met this growing problem [51]. Autoantibodies are a crucial part of disease progression in many autoimmune diseases, and CAAR T cell therapy targets the treatment of these diseases by targeting this important causative aspect of autoimmune diseases. CAAR T cell therapy is only in its infancy of the potential it could have on treatment autoimmune diseases moving into the future. The use of bispecific LINK CAAR T cells as described herein, when applied to desired and applicable causative cell markers, promises novel treatment protocols.

As discussed, GD pathogenesis is mediated by autoreactive B cells specific for the thyroid protein TSHR. The TRAbs produced by these cells bind to TSHR, inducing hyperthyroidism by chronically stimulating hormone production. Current treatments for GD require lifelong treatment or have significant side effects, and none of these available treatments address the anti-TSHR B cells which are crucial to the pathogenesis of GD. A treatment is needed that can selectively eliminate the anti-TSHR B cells from GD patients, stopping production of TRAbs, and halting disease progression without causing significant side effects to the patient.

CAAR T cells can be developed to eliminate anti-TSHR B cells by expressing an epitope of TSHR as the binding domain of the CAAR T cell to act as bait for the anti-TSHR B cells. As the anti-TSHR B cells bind to the epitopes of TSHR on the CAAR T cell, the binding interaction will stimulate CAAR T cell cytotoxicity and will allow CAAR T cell elimination of autoreactive B cells. This may be accomplished through first selecting epitopes of TSHR for the binding domain and engineering the CAAR constructs. Then, the ability of TSHR to serve as the binding domain of the CAAR is characterized by evaluating its binding to TRAbs. B cell lines may be engineered to mimic anti-TSHR B cells found in GD patients.

The cytotoxic function of the CAAR T cells is evaluated against an engineered anti-TSHR B cell line by measuring cytotoxicity, proliferation, and cytokine release. To evaluate the effect of soluble TRAbs and TSH on the engineered CAAR T cells, the cytotoxicity assays may be repeated in the presence of soluble TRAbs and TSH. Finally, bispecific LINK CAR technology will be applied to the engineered CAAR T cells. A TSHR epitope and CD19 scFv will serve as the two binding domains with SLP-76 and LAT as the signaling domains. These engineered LINK CAAR T cells may then be compared to the original CAAR T cells to evaluate their cytotoxicity and specificity.

The therapy will harness the specific killing power of CAR T cells to eliminate the autoreactive B cells of GD. Application of novel bispecific LINK CAR technology will ideally make CAAR T cell treatment of GD increasingly safe and effective. Thus, CAAR T cells could provide a potentially curative treatment to GD patients, filling a need for a more effective treatment option.

### Engineering of a Panel of CAARs With Varying Epitopes of TSHR as the Binding Domain and Evaluating Their Surface Expression and Activation in Jurkat T Cells

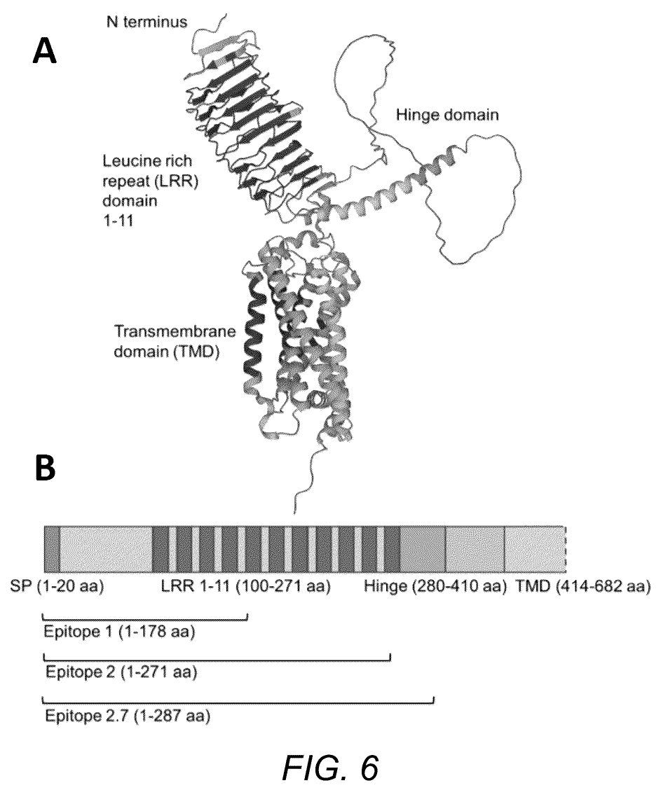

TSHR epitope selection for CAAR binding domain: To engineer a CAAR T cell for treatment of GD, the binding region was evaluated to determine the best options. Epitopes of TSHR are a central component of the engineered CAAR T cells, as they are the binding domain which allows for specific targeting of anti-TSHR B cells. To select the epitopes predicted to fold autonomously and bind most effectively to TRAbs and anti-TSHR B cells, TSHR was first modeled using AlphaFold2 (see FIG. 6 at A, showing the various domains). Epitopes were selected to include the majority of the LRR domain, as this is the region where disease-causing, stimulating TRAbs bind. Epitopes were also selected to have varying sizes around the size of typical CAR binding domains. Epitopes 1-3 were selected and modeled on AlphaFold2 to predict whether they will fold autonomously, and Epitope 3 was subsequently modified to Epitope 2.7 to optimize folding to proper conformation (see FIG. 6 at B, showing Epitope 1 (1-178 amino acids (aa)), Epitope 2 (1-271 aa) and Epitope 2.7 (1-287 aa)). DNA fragments for each epitope were synthesized at TwistBioscience® with a GS linker after the epitope sequence and a BpiI restriction site at each end to facilitate ease of cloning into the CAAR constructs as the binding domain.

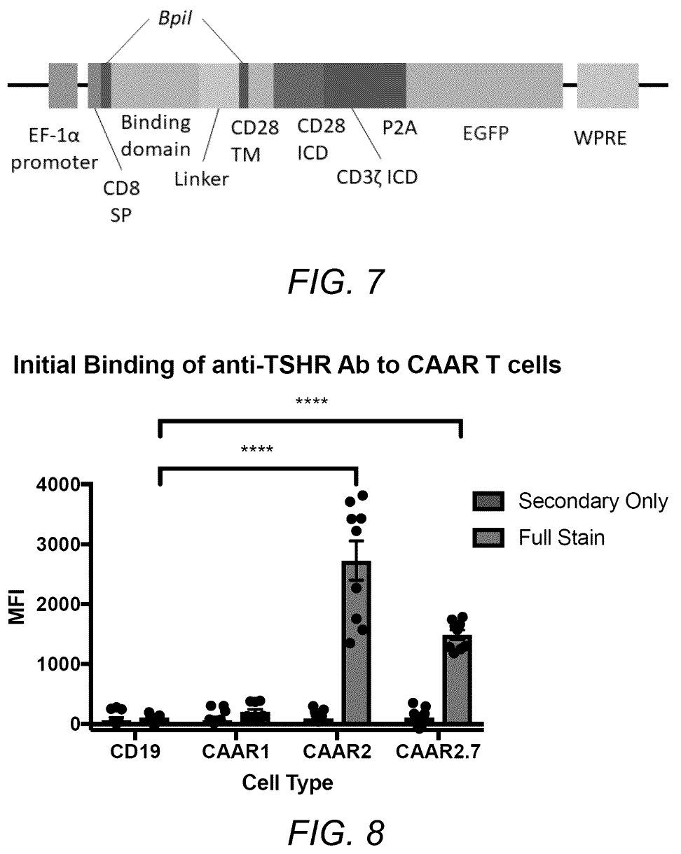

CAAR construct plasmid cloning and transfection conformation: The creation of the engineered CAAR construct started with a base plasmid (Addgene #135991) which is a standard CAR vector containing an extracellular binding domain and linker, CD28 transmembrane domain, and CD28 and CD3ζ signaling domains, as illustrated in FIG. 7 (structure is annotated: the left side contains the CAAR under the same promoter as EGFP, but separated by a self-cleaving peptide, P2A; the right side contains genes for replication in bacteria as well as lentiviral production). This base CAR plasmid was selected for beginning, proof-of-concept work with CAAR T cells, though different signaling domains and other tweaks to the CAR could follow with further work into CAAR T cells. Some changes to the CAR construct could include LINK CAR technology, including alternate activation domains so the CAAR T cells only activate if binding to both TSHR and CD19, a common B cell marker, ensuring the strength of the cytotoxic reaction along with specificity of the LINK CAAR T cells. The plasmid also includes EGFP after the CAR separated by P2A. The synthesized epitope fragments were also digested, purified, and cloned into the base plasmid at the BpiI sites to create the panel of CAAR constructs: CAAR1, CAAR2, and CAAR2.7. Each construct was sequence verified to ensure proper cloning. Constructs were transfected into HEK293T cells to ensure proper expression of the CAAR. After 24 hours cells were analyzed using flow cytometry, and EGFP expression showed that the CAAR construct was being effectively translated (data not shown).

Lentiviral particle fabrication containing CAAR constructs and transduction into a Jurkat T cell line: To allow for stable integration of the CAAR constructs into T cells, and thus creating CAAR T cells, lentiviral particles needed to be produced to transduce the constructs into cells. HEK293FT cells were co-transfected with the CAAR plasmid and lentiviral packaging and envelope plasmids (pMD2.g, pRSV, and PMDL). Lentiviral supernatant was collected, filtered, and ultracentrifuged to isolate pure lentivirus for the CD19 CAR plasmid and each CAAR construct in the panel. HEK293FT cells were transduced with each virus, and the viral titer was determined. Transduced cells were analyzed using flow cytometry, where EGFP expression showed efficient transduction in correlation to the amount of virus added. To create stable cells lines for each of the engineered CAAR T cells that could be used for binding and activation assays, a Jurkat T cell line was transduced with each virus. The Jurkat line is the most established T cell line in CAR research for preliminary studies. EGFP expression averaged between 50-70% for each construct, indicating significant transduction of Jurkat cells with the engineered CAAR-containing lentivirus with high enough efficiency to run further binding and activation assays. Lentivirus was produced and used following proper biosafety level II guidelines, and as approved by the Institutional Biosafety Committee.

Initial binding tests of CAAR T cells to TRAbs: A crucial first test to determine the potential efficacy of our TSHR CAAR T cells as a treatment for GD was to evaluate the function of the selected TSHR epitopes as the binding domain of the CAAR. TRAb binding is highly dependent on the conformation of the TSHR LRR domain, thus a determination was made if the conformation was preserved as epitopes of TSHR were incorporated into the engineered CAAR T cell as the binding domain.

Binding of each CAAR T cell was evaluated using flow cytometry. The primary stain was an anti-TSHR monoclonal Ab (clone M22), derived from GD patient serum and is representative of stimulating TRAbs [9,52]. An untransduced control was used to assist in gating and a CD19 CAR T cell control was performed. CAAR1 did not show significant binding, but both CAAR2 and CAAR2.7 significantly bound to the M22 anti-TSHR Ab, as illustrated in FIG. 8. FIG. 8 shows flow cytometry binding data of the CD19, CAAR1, CAAR2, and CAAR2.7 constructs. Cells were stained with anti-TSHR M22 Ab and a PE-conjugated secondary Ab. Three biological replicates were run, each with three separate technical replicates. Medial fluorescence intensity (MFI) on the y axis with cell type on the x axis. A one-way ANOVA showed CAAR2 and CAAR 2.7 were significantly different from the CD19 control (p<0.0001). CAAR 1 did not show significant binding.

CAAR2 average binding is higher than CAAR2.7. It is hypothesized that CAAR2 could have better binding than CAAR2.7 because CAAR2 includes the entirety of the LRR domain that is key for TRAb binding, while being the size of a normal CAR T cell binding domain (˜275aa). In some embodiments, the epitope has a binding domain sized between approximately 250 and approximately 300 aa, while in other embodiments, the epitope has a binding sized in a range of approximately 250-approximately 295aa, approximately 250-approximately 290aa, approximately 250-approximately 285aa, approximately 250-approximately 280aa, approximately 250-approximately 275aa, approximately 255-approximately 300aa, approximately 255-approximately 295aa, approximately 255-approximately 290aa, approximately 255-approximately 285aa, approximately 255-approximately 280aa, approximately 255-approximately 275aa, approximately 260-approximately 300aa, approximately 260-approximately 295aa, approximately 260-approximately 290aa, approximately 260-approximately 285aa, approximately 260-approximately 280aa, approximately 260-approximately 275aa, approximately 265-approximately 300aa, approximately 265-approximately 295aa, approximately 265-approximately 290aa, approximately 265-approximately 285aa, approximately 265-approximately 280aa, approximately 265-approximately 275aa, approximately 270-approximately 300aa, approximately 270-approximately 295aa, approximately 270-approximately 290aa, approximately 270-approximately 285aa, approximately 270-approximately 280aa, approximately 270-approximately 275aa, approximately 275-approximately 300aa, approximately 275-approximately 295aa, approximately 275-approximately 290aa, approximately 275-approximately 285aa, approximately or 275-approximately 280aa, or ranges falling therein. The hinge region included in epitope 2.7 could also be affecting the folding of the TSHR epitope as it is incorporated in the CAAR, leading to lower levels of TRAb binding, or could have lower expression levels because it is bigger than epitope 2.

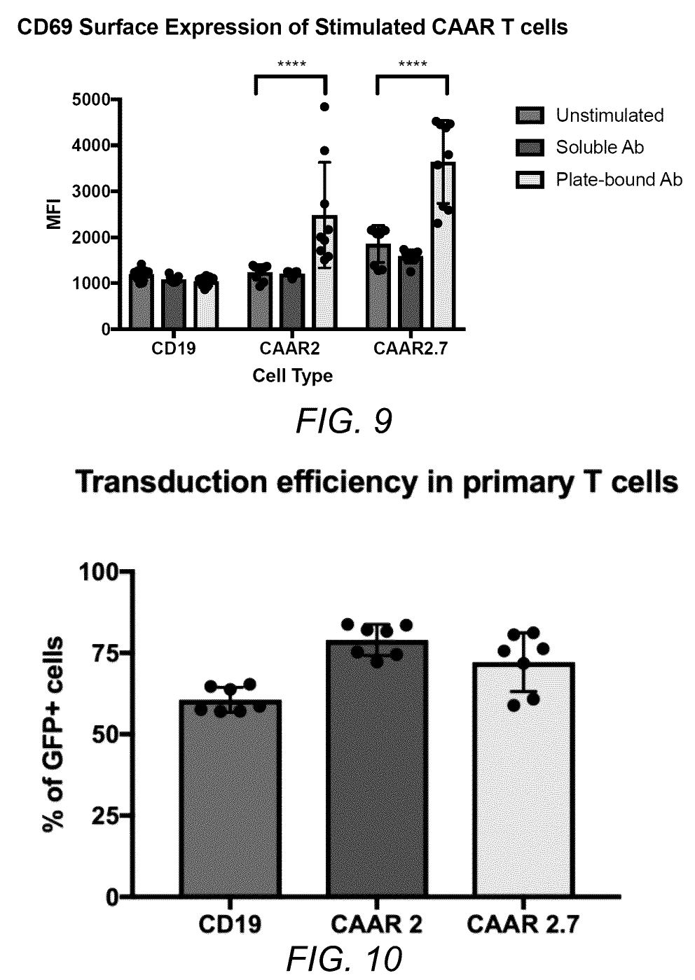

Preliminary activation assays: To determine if this binding transmits an activation signal, Jurkat cells were used. Jurkat cells do not have cytotoxic abilities; however, it is common to measure CD69 expression as an indicator of activation in CAR T cell research. An unstimulated control was used to compare CD69 expression due to activation to basal expression of CD69. Stimulation was performed with soluble and plate-bound anti-TSHR (M22) Ab. As expected, CD19 expressing CAR T cells did not have an increase in CD69 expression after either stimulation condition. Soluble Ab did not significantly activate the CAAR2 and CAAR2.7 T cells, though plate bound stimulation did significantly activate CAAR2 and CAAR 2.7, as illustrated in FIG. 9, which shows initial flow cytometry CD69 activation data. Jurkat cells transfected with CAAR constructs were stimulated with soluble anti-TSHR (M22) Ab or with plate bound M22 Ab. Two biological replicates were run, each with three technical replicates. An untransduced control was used to gate for EGFP expressing cells as before. CD69 levels were measured by flow cytometry as a measurement of cell activation. The CAAR2 and CAAR2.7 constructs stimulated with plate bound Ab were activated in a statistically significant manner compared to activation with soluble Ab and unstimulated cells (p<0.0001) when measured by one-way ANOVA.

Thus, when co-incubated with plate bound stimulatory anti-TSHR Ab, the engineered T cells expressing the TSHR bait are activated. It is hypothesized that CD69 upregulation was significant with plate bound Ab because it more closely simulates a cell-to-cell interaction and provides the concentration of Ab needed for activation. It is beneficial for therapeutic efforts that soluble anti-TSHR Ab does not activate the engineered CAAR T cell, because soluble Abs will be present in GD patients and if it were to activate the CAAR T cells it could overstimulate the CAAR T cells and lead to hyperinflammation or other negative side effects. However, it is possible that the soluble anti-TSHR Abs will provide competition for CAAR T cell binding to the anti-TSHR B cells, thus the effect of soluble anti-TSHR Ab competition should be evaluated further.

Evaluate the cytotoxic abilities of primary CAAR T cells against engineered anti-TSHR B cell lines: The key to evaluating the efficacy of the engineered CAAR T cells as a potential treatment for GD is measuring their ability to specifically eliminate anti-TSHR B cells. To approximate how this treatment would work in a GD patient as closely as can be done in vitro, the cytotoxicity assays are performed using CAAR T cells engineered in primary human T cells. Two anti-TSHR B cell lines were also engineered that have BCRs based on two stimulating TRAbs that have been isolated from GD patients. This provides two physiologically relevant target cell lines to evaluate CAAR T cell killing of anti-TSHR B cells. Several assays may be run to characterize the activation, killing potential, and proliferation of the engineered CAAR T cells when co-incubated with the anti-TSHR B cell lines. Cytometric bead arrays may be used to quantify release of proinflammatory cytokines, flow cytometry proliferation assays measure CAAR T cell proliferation, and cytotoxicity assays visualize and quantify elimination of anti-TSHR B cells. This combination of assays provides a well-rounded picture of the capability of the engineered CAAR T cells to eliminate anti-TSHR B cells compared to B cells without anti-TSHR BCRs.

Transduction of primary human T cells with CAAR constructs: Primary human T cells exhibit complete cytotoxic capacity unlike the Jurkat cell line used in the preliminary studies; thus, to perform cytotoxicity and stimulation assays on the engineered CAAR T cells, the assays need to be run using CAARs engineered into primary human T cells. Blood was drawn from donors under a protocol approved by an Institutional Review Board. PBMCs were isolated using BD Vacutainer CPT™ tubes, then T cells were isolated using the EasySep™ human CD8⁺ T cell isolation kit. T cells were cultured in Primary human T cell media with CD3/CD28 Dynabeads™ and supplemented with IL-2 to activate them and promote proliferation and longevity of the T cells. After 48 hours of activation, the T cells were transduced with CAAR lentivirus calculated for an MOI of 2 and 8 μg/ml of polybrene. Consistent transduction rates of over 70% were achieved across different donor T cells, as illustrated in FIG. 10, which shows flow cytometry data showing transduction efficiencies of the different CAR lentivirus into primary human T cells. Transduction efficiencies were consistently >70%, ranging from 70%-82%. Four biological replicates are shown in FIG. 10 (3 in duplicate, 1 single). Transduction efficiency was measured by GFP positivity, as the CAR constructs include GFP. After 7 days of activation with the CD3/CD28 beads, the cells were removed from the beads, washed, and cultured in media without IL-2 supplementation for 24 hours before beginning any functional assays.

Engineer anti-TSHR B cell lines: The B cell lines engineered to be the target cells for further evaluation of the engineered CAAR T cells should approximate GD patient autoreactive B cells as closely as possible to give the most accurate evaluation of CAAR T cell efficacy. The engineered B cell lines express BCRs with variable sequences derived from TRAbs isolated from GD patients (M22 and K1-18) [52,53].

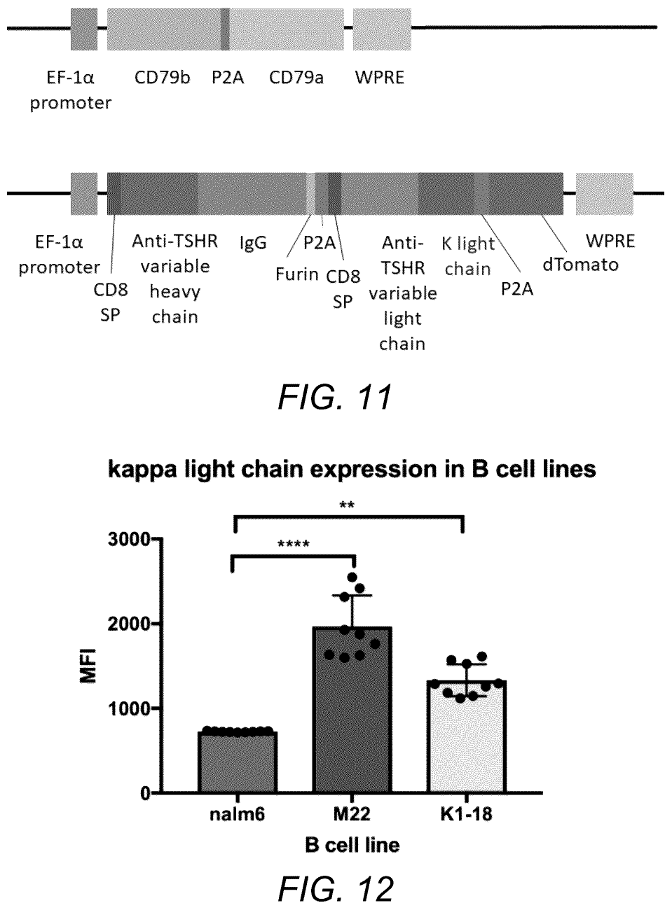

These Abs have been mapped to TSHR and show binding epitopes characteristic of binding epitopes across GD patients [9]. CAAR2 and CAAR2.7 include the entirety of the TSHR leucine rich repeat domain, where all stimulating TRAbs bind, thus it was expected that all stimulating TRAbs and the B cells that produce them would bind to the TSHR epitope on the surface of the engineered CAAR T cells. nalm6 cells were first selected, a lymphoblastic leukemia cell line that expresses CD19. Two plasmids were engineered to equip the nalm-6 cells with anti-TSHR BCRs, the first with BCR costimulatory domains, and the second with anti-TSHR surface immunoglobulin. For the first plasmid CD79a and CD79b are expressed in a lentiviral vector, separated by a P2a site, as shown in FIG. 11. FIG. 11 illustrates simplified plasmid maps for engineered anti-TSHR B cell lines. Above in FIG. 11, an illustrative B cell coreceptor plasmid with CD79b and CD79a separated by a P2A site. Below in FIG. 11, an illustrative anti-TSHR BCR plasmid with CD8 signal peptides (SP) before each anti-TSHR variable region. Each chain complex is separated by P2A. dTomato is expressed in the same transcript, also separated by P2A. The second set of plasmids includes the variable sequence for TRAbs that are derived from GD patients and are characteristic of TRAbs across GD patients.

The M22 and K1-18 Ab variable heavy and light chains, and the IgG constant region and kappa light chain were cloned into a lentiviral vector along with dTomato. These plasmids were cloned by VectorBuilder™. Lentivirus was produced for each plasmid, transduced into nalm6 cells, and the FACS sorted for the fluorescent marker and CD79 expression to create cell lines with 100% expression of the coreceptor and BCR plasmids. After expansion of the cell lines, they were validated by staining with an anti-kappa light chain Ab, to confirm expression of the BCRs and both M22 and K1-18 have significant expression, as shown in FIG. 12. FIG. 12 shows a measure of BCR expression in engineered anti-TSHR B cell lines. M22 and K1-18 express significant levels of BCRs. nalm6 cells do not express any BCRs, so kappa light chain expression signifies conformational expression of the BCR following lentiviral transduction.

CAAR T cell/anti-TSHR B cell co-incubation for cytokine production, cytotoxicity, and proliferation analyses: Cytokine production, cytotoxicity, and proliferation are critical ways to measure T cell activation and cytotoxicity and provide a quantifiable way to evaluate the efficiency of the engineered CAAR T cells at killing autoreactive B cells. For the flow cytometry based cytotoxicity assay, each of the CAAR T cells was co-incubated with each of the engineered anti-TSHR B cell lines and untransduced nalm6 B cells, and flow cytometry was run at 0, 12, 24, and 48 hours to count the number of live CAAR T cells (using their EGFP fluorescence, and the number of live B cells, using their dTomato fluorescence), to quantify the killing and expansion that takes place. At 24 and 48 hours, the supernatant was saved from the coincubation assay, and a cytometric bead array (CBA) was performed to quantify release of proinflammatory cytokines, including IFNγ, TNF, IL-2, and IL-6.

The cytotoxicity of the engineered CAAR T cells against the B cell lines was directly measured by co-incubating them in the ImageExpress® Pico for a period of 48 hours. This machine images every hour and counts the number of CAAR T cells and anti-TSHR B cells by detection of EGFP and RFP. This allows measuring the killing of the anti-TSHR B cells through fluorescent microscopy and cell counting, giving qualitative images as well as a more detailed quantitative cell counts over time. Finally, a CFSE proliferation assay was performed on the CAAR T cells incubated with the B cells, using flow cytometry after 12, 24, and 48 hours to measure the proliferation of the CAAR T cells expected upon activation. All the previously described experiments were run with an untransduced T cell negative control as well as the CD19 CAR T cell positive control. There were also samples of CAAR T cells alone and each B cell line alone to get a baseline. Each experiment was run with three biological replicates, each in triplicate.

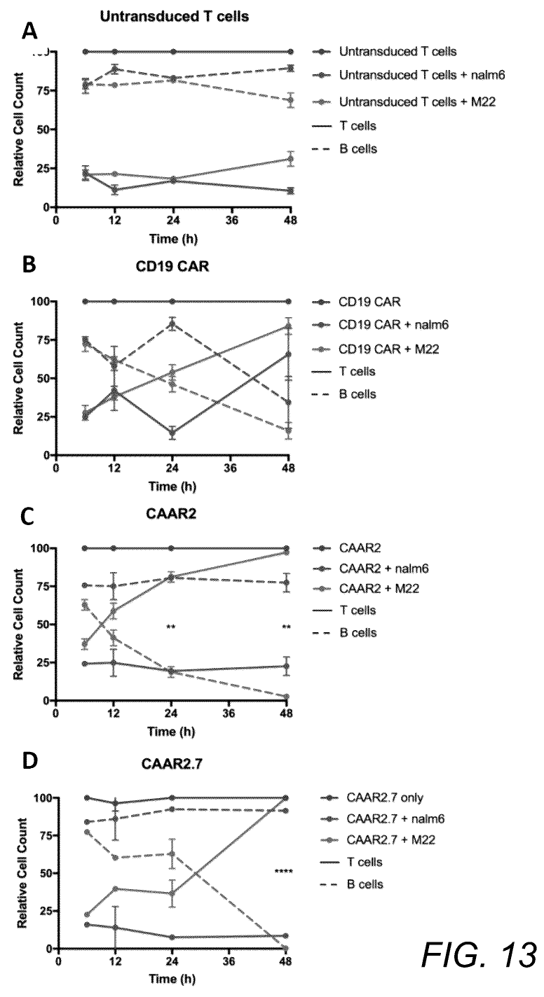

Outcomes and alternative methods: a preliminary flow cytometry cytotoxicity assay was performed to ensure that targeted cytotoxicity by the engineered CAAR T cells could be see, and the preliminary data show, as expected, that untransduced T cells have no cytotoxicity, as shown in FIG. 13 at A, and the CD19 CAR specifically killed all B cells, as shown in FIG. 13 at B. FIG. 13 shows preliminary flow cytometry cytotoxicity data from experiments in which 8,000 T cells and B cells were co-incubated, and flow cytometry was run at four time points to count the number of alive T cells and B cells. T cells are shown in solid lines and B cells in dashed lines. T tests were run comparing the ratio of B cells to T cells under each co-incubation pair at each time point (**p<0.001, ****p<0.0001).

CAAR2 (FIG. 13 at C) and CAAR2.7 (FIG. 13 at D) demonstrated efficient cytotoxicity against the M22 B cell line by 48 hours, without killing the nalm6 B cells. This CAAR cytotoxicity against M22 was a stronger response than the CD19 CAR against the different B cell lines, likely because the anti-TSHR BCR expression was expressed at a higher level than CD19. This preliminary experiment was run with only one biological replicate in duplicate, but more replicates will be performed later. It is expected that the engineered CAAR T cells, when co-incubated with the engineered B cell lines, will have significant production of IFN-γ, TNF, IL-2, and IL-6, and significantly increased proliferation when compared with the untransduced T cell control. The CAAR T cells incubated with the engineered anti-TSHR B cells should have comparable levels of proinflammatory cytokine production and proliferation as the CD19 CAR T cells incubated with any of the B cell samples, because the engineered B cell line expresses CD19. Identification of cell concentrations and assay timepoints to ensure proper readouts on positive and negative controls is needed.

It is possible that because both the CAAR T cells and anti-TSHR B cells are in suspension, the ImageExpress® Pico may have difficulty focusing on the cells over the period of the 48 hours. This may not affect the data greatly, but if it does, a SpecrtaMax® iD3 fluorometer can be used to analyze the quantity of green and red fluorescence. This should give comparable quantifiable data that will be consistent as it does not require a microscope to focus overtime; however, it does not provide the images at each time point.

Evaluation CAAR T cell interaction with anti-TSHR B cells in the presence of TRAbs and TSH: CAAR T cell treatment is to be given intravenously to a patient, and thus encounters many more cell types and molecules in vivo than the previously described in vitro experiments with only CAAR T cells and their target anti-TSHR B cells. Particular attention has been given to soluble TSH that may interact with the epitopes of TSHR on the surface of the engineered CAAR T cells. This has the potential to cause off-target effects or to bind a significant amount of TSH in the body and cause a hormone imbalance. Thus, a determination should be made if TSH binds with the engineered CAAR T cells and, if so, the physiological effect thereof.

The role of soluble TRAbs should also be evaluated. Preliminary data indicates they will bind to the engineered CAAR T cells, and a determination should be made if this will have an inhibitory effect, neutral effect, or enabling effect on the cytotoxicity of the engineered CAAR T cells. Data also shows that soluble anti-TSHR Abs do not activate the CAARs unless the Ab is plate bound. In experiments with CAAR T cells in other diseases, results concluded that soluble autoantibodies did not have a significant negative impact on CAAR T cell function, and in some cases even had a slight beneficial effect [44,46,47]. Though it is hoped that soluble TRAbs will not activate the CAAR T cells, there still is a potential for them to activate, possibly leading to a cytokine storm or exhaustion of the CAAR T cells. Even if the soluble TRAbs do not activate the CAAR T cells, they could compete with the anti-TSHR B cells for binding to the TSHR epitopes of the CAAR and prevent some of their function, so an evaluation of the cytotoxicity of the engineered CAAR T cells in the presence of physiological levels of commercially available TRAbs as well as GD patient serum is planned.

Off-target cytotoxicity analysis and inhibition in presence of TSH and soluble TRAbs: The epitopes of TSHR on the engineered CAAR T cells have the potential to cross-react with TSH found naturally in the body. If the TSH significantly activates the CAAR T cells, it could produce chronic inflammation. Thus, a determination will be made if TSH will bind to and activate the engineered CAAR T cells. First, flow cytometry binding analysis using TSH and a fluorescently conjugated secondary Ab that binds with the tag on TSHR will be run to determine if there is binding of TSH to the engineered CAAR T cells. Then, IFN-y production will be measured using the supernatant after co-incubation of the CAAR T cells and TSH. The cytotoxicity experiments will then be repeated performed in the presence of TSH at concentrations of 0.5 mIU/L and 4.5 mIU/L (the low and high end of normal TSH levels in blood). GD patients often have lower than normal levels of TSH because of the negative feedback loop caused by hyperthyroidism, but these TSH levels should provide a picture of likely circumstances in vivo. This will allow determination if TSH is a potential problem in off-target cytotoxicity.

Similarly, it is anticipated that soluble TRAbs will bind to the engineered CAAR T cells, and a determination will be made if this will inhibit CAAR T cell function or have a neutral effect. To determine which of these will be the case, the cytotoxicity assays described above will be performed in the presence of commercially available TRAbs in concentrations representative of mild, moderate, and severe GD, 1.75 IU/L, 10 IU/L, and 40 IU/L, respectively. Cytotoxicity experiments will also be performed again in the presence of GD patient serum from at least three patients, to further conclude the effect of soluble TRAbs on CAAR T cell efficacy.

Expected outcomes and alternative methods: In the attempt to create a safe and effective treatment the effects of TSH and soluble TRAbs on CAAR T cell efficiency should be considered. It is hypothesized that because the regions of TSHR included in selected epitopes are the binding sites for both TRAbs and TSH, some binding to the engineered CAAR T cells may be experienced. However, it will be important to evaluate the effect this has on the cytotoxicity of the CAAR T cells. It will not be possible to fully evaluate the effect of CAAR T cells on TSH levels with in vitro experiments; however, evaluation will continue in in vivo experimentation.

Again, it is expected that TRAbs bind to the engineered CAAR T cells and a determination will be made if this will have a significant effect on the ability of the engineered CAAR T cells to eliminate anti-TSHR B cells. Ideally there will not be a significant negative effect on CAAR function, and there may even be a beneficial effect; however, some CAAR T cell inhibition is a possibility. Inhibition could be related to CAAR T cell exhaustion, in which case, there is research currently ongoing on metabolic and signaling modifications that can equip CAR T cells with tools to resist exhaustion. It is possible that some of these new findings could be applied to the engineered CAAR T cells as well, should this problem be encountered. Having a panel of CAAR constructs will provide alternatives that may perform better when exposed to soluble TRAbs and TSH. GD patient serum will be purchased from the Mayo Clinic or sourced from GD patient volunteers.

Apply bispecific LINK CAR technology to GD CAAR T cells and compare their efficacy and specificity to original CAAR T cells: CAAR T cells are a novel way to treat autoantibody-mediated autoimmune diseases, but the incorporation of a naturally occurring protein as the surface binding domain could lead to off-target effects and make the treatment less safe. It is hypothesized that bispecific LINK CAR application has the potential to eliminate any possible off-target effects by requiring binding of two antigens to activate the CAR T cell. LINK CAAR constructs for treatment of GD will be developed with a TSHR binding domain and an anti-CD19 scFv binding domain and will be transduced into T cells to create bispecific LINK CAAR T cells [33]. They will be evaluated for their cytotoxicity and specificity at eliminating only anti-TSHR B cells. They will be compared to the original engineered CAAR T cells for GD to determine if the specificity is improved. This application has the potential of making CAAR treatment of autoimmune diseases safer and more effective by reducing the risk for potential cytokine storms and increasing the specificity of cytotoxicity. It could also mitigate potential problems associated with competition of soluble TRAbs and the anti-TSHR B cells.

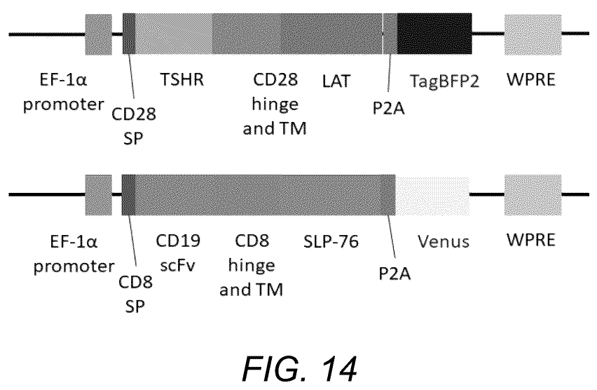

LINK CAAR design and production: LINK CAR technology enables improved specificity of a CAR T cell. This is achieved by altering the signaling domains and using downstream signaling molecules (SLP-76 and LAT) as the activation domains. In the application to CAAR T cells for treatment of GD one CAR construct has a TSHR binding domain and the other has an scFv which binds to CD19. The signaling, hinge, and transmembrane domains are different in the two CARs to prevent dimerization and potential activation when only one antigen is bound. The constructs have different fluorescent proteins enabling detection of each CAR after transduction into T cells, as illustrated in FIG. 14, which provides an exemplary LINK CAAR constructs map. TSHR and a CD19 scFv are the binding domains and the activation domains are LAT and SLP-76. Activation of both is necessary for T cell activation. TagBFP2 and Venus were selected because they exhibit minimal spectral overlap, increasing their ability to be distinguished from each other.

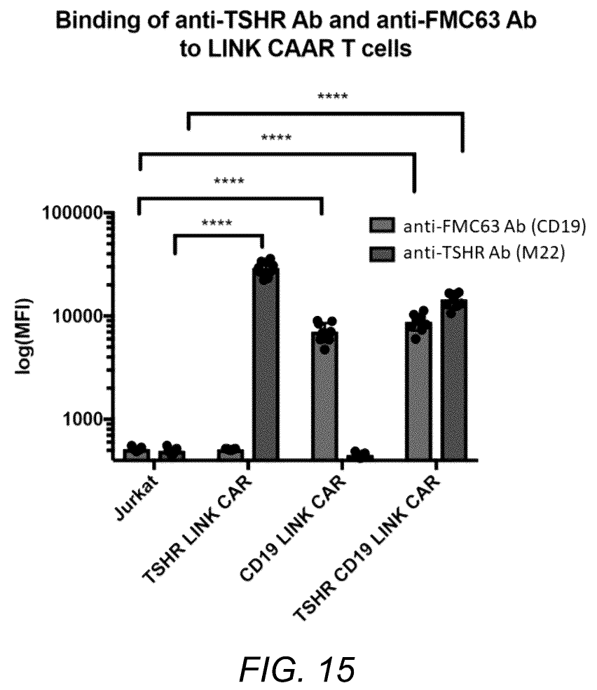

These LINK CAAR constructs were cloned by VectorBuilder™ and then sequence confirmed. Lentiviral particles were fabricated following the same procedure as previously described. Jurkat cells were transduced with the plasmids, separately and combined, to confirm that the CARs transduce effectively, and then FACS sorted the cells to create stable cell lines of each LINK CAAR. Flow cytometry binding assays were performed to ensure proper binding to an anti-CD19 scFv Ab (FMC63) and to TRAbs (M22) and they worked as expected, as illustrated in FIG. 15. FIG. 15 shows expression and binding of LINK CAR T cells in Jurkat cells to their specific antigen. Jurkat cells with only one of the LINK constructs bound only to their antigen (anti-CD19 scFv bound by anti-FMC63 Ab, TSHR bound by M22 Ab), while the cells engineered with both LINK constructs bound to both. Three biological replicates, each with three technical replicates (p<0.0001, measured by one way ANOVA).

Cytotoxicity analysis of LINK CAAR T cells in comparison with the original CAAR T cells: To evaluate the cytotoxicity of the LINK CAAR T cells, they will be transduced into primary human T cells, using the methods previously described. The anti-TSHR B cell lines previously engineered will serve as the primary target cell line, but untransduced nalm6 cells will serve as a CD19+/anti-TSHR Ab-control, and CRISPR/Cas9 will be used to knock out CD19 in the anti-TSHR B cell lines for a CD19−/anti-TSHR+ control. This will allow detection of any possible off-target cytotoxicity when only one of the two antigens is bound. The cytotoxicity assays will also be repeated to quantify the strength and speed of the cytotoxic reaction when the LINK CAAR T cells are co-incubated with anti-TSHR B cells (which also express CD19). These results will be compared with the original CAAR T cell data to determine if the LINK CAARs are equally as effective at eliminating anti-TSHR B cells, or perhaps even more effective because they bypass some of the steps in the activation process, potentially leading to a faster cytotoxic response.

Specificity analysis of bispecific LINK CAAR T cells in comparison with the original CAAR T cells: it is hypothesized that the LINK CAAR T cells will be more specific than the original CAAR T cells because of their bispecificity. To evaluate this, the CD69 activation assay, the proliferation assay, and cytometric bead array measurement of cytokine release when incubated with plate bound anti-TSHR Ab and TSH stimulation will be repeated. This will help to understand if these soluble factors can cause off-target activation. Cytotoxicity experiments co-incubated with anti-TSHR B cells in the presence of anti-TSHR Ab and TSH will also be repeated. These results will be compared with the previous results to determine if the LINK CAAR T cells have increased specificity. Flow cytometry will also be used to compare expression of exhaustion markers (CTLA4, PD-1, TIM3 and LAG3) after stimulation with anti-TSHR B cells for one week, to determine if the LINK CAAR T cells are less prone to exhaustion than the original CAAR T cells. Previous research on LINK CAR T cells shows that they exhibit lower levels of exhaustion markers than traditional CAR T cells [33].

Expected outcomes and alternative methods: It is expected that the LINK CAAR T cells will exhibit high binding affinity for THSR and CD19 in the respective constructs. When testing for activation in Jurkat cells using plate bound Ab, activation of the full LINK CAAR is expected only when incubated with anti-TSHR Ab and CD19 beads both plate-bound together. Plates with one of the antigens plate-bound should not activate the LINK CAAR T cell. Conversely T cells transduced with only one of the CARs in the LINK CAAR system should not be activated by any combination of plate bound antigens. The evaluation and comparison of LINK CAAR T cells to the original CAAR T cells will be informative. It is expected that there will be a difference in the specificity where no activation occurs when LINK CAAR T cells are stimulated with TRAbs; however, it is anticipated that a high titer of TRAbs would likely activate the original CAAR T cells.

Whether this is detrimental to the efficacy of the CAAR T cells remains to be seen, though it is believed this could lead to exhaustion of the CAAR T cells. Thus, the experiments monitoring exhaustion surface markers after prolonged stimulation will be important to run. It is expected that the LINK CAAR T cells will outperform the original CAAR T cells in terms of prolonged activation without significant levels of exhaustion markers. Based on the data received, it could also be beneficial to run another cytometric bead array to evaluate the changes in cytokine release levels over time to see if the activation state over time is different between the LINK CAAR T cells and the original CAAR T cells. Initial exhaustion tests will be run after seven days of stimulation, but after seeing the initial data, these experiments may need to be run at multiple time points. One possible limitation of this therapy is the persistence of plasma cells, which could happen because they downregulate expression of CD19 and BCRs. It is hoped that because of the persistence of CAR T cells, even though plasma cells express low levels of these proteins, over time they will still be eliminated by the LINK CAAR T cells. However, if this proves not to be the case, one possible solution is to swap the anti-CD19 scFv for an anti-BCMA scFv, because plasma cells typically express BCMA at higher levels than CD19.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

### REFERENCES

1. Smith, T.J.; Hegedüs, L. Graves' Disease. N Engl J Med 2016, 375, 1552-1565, doi:10.1056/NEJMra1510030.
2. Ross, D.S.; Burch, H.B.; Cooper, D.S.; Greenlee, M.C.; Laurberg, P.; Maia, A.L.; Rivkees, S.A.; Samuels, M.; Sosa, J.A.; Stan, M.N.; et al. 2016 American Thyroid Association Guidelines for Diagnosis and Management of Hyperthyroidism and Other Causes of Thyrotoxicosis. Thyroid 2016, 26, 1343-1421, doi:10.1089/thy.2016.0229.
3. Bartalena, L.; Piantanida, E.; Gallo, D.; Ippolito, S.; Tanda, M.L. Management of Graves' hyperthyroidism: present and future. Expert Rev Endocrinol Metab 2022, 17, 153-166, doi:10.1080/17446651.2022.2052044.
4. Hansen, M.; Johnson, A.; Weber, K.S.; O'Neill, K.L. Characterizing the Interplay of Lymphocytes in Graves' Disease. Int J Mol Sci 2023, 24, doi:10.3390/ijms24076835.
5. Zhou, F.; Wang, X.; Wang, L.; Sun, X.; Tan, G.; Wei, W.; Zheng, G.; Ma, X.; Tian, D.; Yu, H. Genetics, Epigenetics, Cellular Immunology, and Gut Microbiota: Emerging Links With Graves' Disease. Front Cell Dev Biol 2021, 9, 794912, doi:10.3389/fcell.2021.794912.
6. Myshunina, T.M.; Guda, B.D.; Bolgov, M.Y.; Mikhailenko, N.I.; Tronko, N.D. Differentiated thyroid carcinomas associated with chronic thyroiditis: biological and clinical properties. Exp Oncol 2018, 40, 128-131.
7. Belfiore, A.; Garofalo, M.R.; Giuffrida, D.; Runello, F.; Filetti, S.; Fiumara, A.; Ippolito, O.; Vigneri, R. Increased aggressiveness of thyroid cancer in patients with Graves' disease. J Clin Endocrinol Metab 1990, 70, 830-835, doi:10.1210/jcem-70-4-830.
8. Palella, M.; Giustolisi, F.M.; Modica Fiascaro, A.; Fichera, M.; Palmieri, A.; Cannarella, R.; Calogero, A.E.; Ferrante, M.; Fiore, M. Risk and Prognosis of Thyroid Cancer in Patients with Graves' Disease: An Umbrella Review. Cancers (Basel) 2023, 15, doi:10.3390/cancers15102724.
9. Furmaniak, J.; Sanders, J.; Núñez Miguel, R.; Rees Smith, B. Mechanisms of Action of TSHR Autoantibodies. Horm Metab Res 2015, 47, 735-752, doi:10.1055/s-0035-1559648.
10. Michalek, K.; Morshed, S.A.; Latif, R.; Davies, T.F. TSH receptor autoantibodies. Autoimmun Rev 2009, 9, 113-116, doi:10.1016/j.autrev.2009.03.012.
11. Morshed, S.A.; Davies, T.F. Graves' Disease Mechanisms: The Role of Stimulating, Blocking, and Cleavage Region TSH Receptor Antibodies. Horm Metab Res 2015, 47, 727-734, doi:10.1055/s-0035-1559633.
12. Akamizu, T.; Kosugi, S.; Kohn, L.D.; Mori, T. [Anti-thyrotropin (TSH) receptor antibody binding epitopes of TSH receptor: site-directed mutagenesis approach]. Nihon Rinsho 1994, 52, 1024-1030.
13. Subekti, I.; Pramono, L.A. Current Diagnosis and Management of Graves' Disease. Acta Med Indones 2018, 50, 177-182.
14. Guedan, S.; Calderon, H.; Posey, A.D.; Maus, M.V. Engineering and Design of Chimeric Antigen Receptors. Mol Ther Methods Clin Dev 2019, 12, 145-156, doi:10.1016/j.omtm.2018.12.009.
15. Moreno, C.; Haynie, C.; Johnson, A.; Weber, K.S. Alternative CAR Therapies: Recent Approaches in Engineering Chimeric Antigen Receptor Immune Cells to Combat Cancer. Biomedicines 2022, 10, doi:10.3390/biomedicines 10071493.
16. Hong, M.; Clubb, J.D.; Chen, Y.Y. Engineering CAR-T Cells for Next-Generation Cancer Therapy. Cancer Cell 2020, 38, 473-488, doi:10.1016/j.ccell.2020.07.005.
17. Holstein, S.A.; Lunning, M.A. CAR T-Cell Therapy in Hematologic Malignancies: A Voyage in Progress. Clin Pharmacol Ther 2020, 107, 112-122, doi:10.1002/cpt. 1674.
18. Kuwana, Y.; Asakura, Y.; Utsunomiya, N.; Nakanishi, M.; Arata, Y.; Itoh, S.; Nagase, F.; Kurosawa, Y. Expression of chimeric receptor composed of immunoglobulin-derived V regions and T-cell receptor-derived C regions. Biochem Biophys Res Commun 1987, 149, 960-968, doi:10.1016/0006-291x(87)90502-x.
19. Gross, G.; Waks, T.; Eshhar, Z. Expression of immunoglobulin-T-cell receptor chimeric molecules as functional receptors with antibody-type specificity. Proc Natl Acad Sci U S A 1989, 86, 10024-10028, doi:10.1073/pnas.86.24.10024.
20. Krause, A.; Guo, H.F.; Latouche, J.B.; Tan, C.; Cheung, N.K.; Sadelain, M. Antigen-dependent CD28 signaling selectively enhances survival and proliferation in genetically modified activated human primary T lymphocytes. J Exp Med 1998, 188, 619-626, doi:10.1084/jem.188.4.619.
21. Maher, J.; Brentjens, R.J.; Gunset, G.; Rivière, I.; Sadelain, M. Human T-lymphocyte cytotoxicity and proliferation directed by a single chimeric TCRzeta/CD28 receptor. Nat Biotechnol 2002, 20, 70-75, doi:10.1038/nbt0102-70.
22. Brentjens, R.J.; Latouche, J.B.; Santos, E.; Marti, F.; Gong, M.C.; Lyddane, C.; King, P.D.; Larson, S.; Weiss, M.; Rivière, I.; et al. Eradication of systemic B-cell tumors by genetically targeted human T lymphocytes co-stimulated by CD80 and interleukin-15. Nat Med 2003, 9, 279-286, doi:10.1038/nm827.
23. Mullard, A. FDA approves first CAR T therapy. Nat Rev Drug Discov 2017, 16, 669, doi:10.1038/nrd.2017.196.
24. Melenhorst, J.J.; Chen, G.M.; Wang, M.; Porter, D.L.; Chen, C.; Collins, M.A.; Gao, P.; Bandyopadhyay, S.; Sun, H.; Zhao, Z.; et al. Decade-long leukaemia remissions with persistence of CD4. Nature 2022, 602, 503-509, doi: 10.1038/s41586-021-04390-6.
25. Neelapu, S.S.; Locke, F.L.; Bartlett, N.L.; Lekakis, L.J.; Miklos, D.B.; Jacobson, C.A.; Braunschweig, I.; Oluwole, O.O.; Siddiqi, T.; Lin, Y.; et al. Axicabtagene Ciloleucel CAR T-Cell Therapy in Refractory Large B-Cell Lymphoma. N Engl J Med 2017, 377, 2531-2544,doi:10.1056/NEJMoa1707447.
26. Abramson, J.S. Anti-CD19 CAR T-Cell Therapy for B-Cell Non-Hodgkin Lymphoma. Transfus Med Rev 2020, 34, 29-33, doi:10.1016/j.tmrv.2019.08.003.
27. Fujiwara, Y.; Kato, T.; Hasegawa, F.; Sunahara, M.; Tsurumaki, Y. The Past, Present, and Future of Clinically Applied Chimeric Antigen Receptor-T-Cell Therapy. Pharmaceuticals (Basel) 2022, 15, doi: 10.3390/ph15020207.
28. Vora, S.B.; Waghmare, A.; Englund, J.A.; Qu, P.; Gardner, R.A.; Hill, J.A. Infectious Complications Following CD19 Chimeric Antigen Receptor T-cell Therapy for Children, Adolescents, and Young Adults. Open Forum Infect Dis 2020, 7, ofaa121, doi:10.1093/ofid/ofaa121.
29. van der Schans, J.J.; van de Donk, N.W.C.J.; Mutis, T. Dual Targeting to Overcome Current Challenges in Multiple Myeloma CAR T-Cell Treatment. Front Oncol 2020, 10, 1362, doi:10.3389/fonc.2020.01362.
30. Kloss, C.C.; Condomines, M.; Cartellieri, M.; Bachmann, M.; Sadelain, M. Combinatorial antigen recognition with balanced signaling promotes selective tumor eradication by engineered T cells. Nat Biotechnol 2013, 31, 71-75, doi:10.1038/nbt.2459.
31. Roybal, K.T.; Williams, J.Z.; Morsut, L.; Rupp, L.J.; Kolinko, I.; Choe, J.H.; Walker, W.J.; McNally, K.A.; Lim, W.A. Engineering T Cells with Customized Therapeutic Response Programs Using Synthetic Notch Receptors. Cell 2016, 167, 419-432.e416, doi:10.1016/j.cell.2016.09.011.
32. Srivastava, S.; Salter, A.I.; Liggitt, D.; Yechan-Gunja, S.; Sarvothama, M.; Cooper, K.; Smythe, K.S.; Dudakov, J.A.; Pierce, R.H.; Rader, C.; et al. Logic-Gated ROR1 Chimeric Antigen Receptor Expression Rescues T Cell-Mediated Toxicity to Normal Tissues and Enables Selective Tumor Targeting. Cancer Cell 2019, 35, 489-503.e488, doi:10.1016/j.ccell.2019.02.003.
33. Tousley, A.M.; Rotiroti, M.C.; Labanieh, L.; Rysavy, L.W.; Kim, W.-J.; Lareau, C.; Sotillo, E.; Weber, E.W.; Rietberg, S.P.; Dalton, G.N.; et al. Co-opting signalling molecules enables logic-gated control of CAR T cells. Nature 2023, 615, 507-516, doi:10.1038/s41586-023-05778-2.
34. Johnson, A.; Townsend, M.; O'Neill, K. Tumor Microenvironment Immunosuppression: A Roadblock to CAR T-Cell Advancement in Solid Tumors. Cells 2022, 11, doi:10.3390/cells11223626.
35. Merrill, J.T.; Neuwelt, C.M.; Wallace, D.J.; Shanahan, J.C.; Latinis, K.M.; Oates, J.C.; Utset, T.O.; Gordon, C.; Isenberg, D.A.; Hsieh, H.J.; et al. Efficacy and safety of rituximab in moderately-to-severely active systemic lupus erythematosus: the randomized, double-blind, phase II/III systemic lupus erythematosus evaluation of rituximab trial. Arthritis Rheum 2010, 62, 222-233, doi:10.1002/art.27233.
36. Gomez Mendez, L.M.; Cascino, M.D.; Garg, J.; Katsumoto, T.R.; Brakeman, P.; Dall'Era, M.; Looney, R.J.; Rovin, B.; Dragone, L.; Brunetta, P. Peripheral Blood B Cell Depletion after Rituximab and Complete Response in Lupus Nephritis. Clin J Am Soc Nephrol 2018, 13, 1502-1509, doi:10.2215/CJN.01070118.
37. Kotagiri, P.; Martin, A.; Hughes, P.; Becker, G.; Nicholls, K. Single-dose rituximab in refractory lupus nephritis. Intern Med J 2016, 46, 899-901, doi: 10.1111/imj.13136.
38. Kansal, R.; Richardson, N.; Neeli, I.; Khawaja, S.; Chamberlain, D.; Ghani, M.; Ghani, Q.U.; Balazs, L.; Beranova-Giorgianni, S.; Giorgianni, F.; et al. Sustained B cell depletion by CD19-targeted CAR T cells is a highly effective treatment for murine lupus. Sci Transl Med 2019, 11, doi:10.1126/scitranslmed.aav1648.
39. Jin, X.; Xu, Q.; Pu, C.; Zhu, K.; Lu, C.; Jiang, Y.; Xiao, L.; Han, Y.; Lu, L. Therapeutic efficacy of anti-CD19 CAR-T cells in a mouse model of systemic lupus erythematosus. Cell Mol Immunol 2021, 18, 1896-1903, doi:10.1038/s41423-020-0472-1.
40. Mackensen, A.; Müller, F.; Mougiakakos, D.; Böltz, S.; Wilhelm, A.; Aigner, M.; Völkl, S.; Simon, D.; Kleyer, A.; Munoz, L.; et al. Anti-CD19 CAR T cell therapy for refractory systemic lupus erythematosus. Nat Med 2022, 28, 2124-2132, doi:10.1038/s41591-022-02017-5.
41. Müller, F.; Boeltz, S.; Knitza, J.; Aigner, M.; Völkl, S.; Kharboutli, S.; Reimann, H.; Taubmann, J.; Kretschmann, S.; Rösler, W.; et al. CD19-targeted CAR T cells in refractory antisynthetase syndrome. Lancet 2023, 401, 815-818, doi:10.1016/s0140-6736(23)00023-5.
42. Mazzi, M.T.; Hajdu, K.L.; Ribeiro, P.R.; Bonamino, M.H. CAR T cells leave the comfort zone: current and future applications beyond cancer. Immunotherapy Advances 2021, Volume 1.
43. Treanor, B. B-cell receptor: from resting state to activate. Immunology 2012, 136, 21-27, doi:10.1111/j.1365-2567.2012.03564.x.
44. Ellebrecht, C.T.; Bhoj, V.G.; Nace, A.; Choi, E.J.; Mao, X.; Cho, M.J.; Di Zenzo, G.; Lanzavecchia, A.; Seykora, J.T.; Cotsarelis, G.; et al. Reengineering chimeric antigen receptor T cells for targeted therapy of autoimmune disease. Science 2016, 353, 179-184, doi:10.1126/science.aaf6756.
45. Hammers, C.M.; Stanley, J.R. Mechanisms of Disease: Pemphigus and Bullous Pemphigoid. Annu Rev Pathol 2016, 11, 175-197, doi:10.1146/annurev-pathol-012615-044313.
46. Lee, J.; Lundgren, D.K.; Mao, X.; Manfredo-Vieira, S.; Nunez-Cruz, S.; Williams, E.F.; Assenmacher, C.A.; Radaelli, E.; Oh, S.; Wang, B.; et al. Antigen-specific B cell depletion for precision therapy of mucosal pemphigus vulgaris. J Clin Invest 2020, 130, 6317-6324, doi:10.1172/JCI138416.
47. Oh, S.; Mao, X.; Manfredo-Vieira, S.; Lee, J.; Patel, D.; Choi, E.J.; Alvarado, A.; Cottman-Thomas, E.; Maseda, D.; Tsao, P.Y.; et al. Precision targeting of autoantigen-specific B cells in muscle-specific tyrosine kinase myasthenia gravis with chimeric autoantibody receptor T cells. Nat Biotechnol 2023, doi: 10.1038/s41587-022-01637-z.
48. Sadeqi Nezhad, M.; Seifalian, A.; Bagheri, N.; Yaghoubi, S.; Karimi, M.H.; Adbollahpour-Alitappeh, M. Chimeric Antigen Receptor Based Therapy as a Potential Approach in Autoimmune Diseases: How Close Are We to the Treatment? Front Immunol 2020, 11, 603237, doi:10.3389/fimmu.2020.603237.
49. Leso, V.; Vetrani, I.; De Cicco, L.; Cardelia, A.; Fontana, L.; Buonocore, G.; Iavicoli, I. The Impact of Thyroid Diseases on the Working Life of Patients: A Systematic Review. Int J Environ Res Public Health 2020, 17, doi:10.3390/ijerph17124295.
50. Duan, H.; Jiang, Z.; Chen, L.; Bai, X.; Cai, H.; Yang, X.; Huang, H. TSHR-based chimeric antigen receptor T cell specifically deplete auto-reactive B lymphocytes for treatment of autoimmune thyroid disease. International Immunopharmacology 2023, 124, 110873, doi:https://doi.org/10.1016/j.intimp.2023.110873.
51. Roberts, M.H.; Erdei, E. Comparative United States autoimmune disease rates for 2010-2016 by sex, geographic region, and race. Autoimmun Rev 2020, 19, 102423, doi:10.1016/j.autrev.2019.102423.
52. Sanders, J.; Jeffreys, J.; Depraetere, H.; Evans, M.; Richards, T.; Kiddie, A.; Brereton, K.; Premawardhana, L.D.; Chirgadze, D.Y.; Núñez Miguel, R.; et al. Characteristics of a human monoclonal autoantibody to the thyrotropin receptor: sequence structure and function. Thyroid 2004, 14, 560-570, doi:10.1089/1050725041692918.
53. Furmaniak, J.; Sanders, J.; Young, S.; Kabelis, K.; Sanders, P.; Evans, M.; Clark, J.; Wilmot, J.; Rees Smith, B. In vivo effects of a human thyroid-stimulating monoclonal autoantibody (M22) and a human thyroid-blocking autoantibody (K1-70). Auto Immun Highlights 2012, 3, 19-25, doi:10.1007/s13317-011-0025-9.

What is claimed and desired to be secured by Letters Patent is:

1. A method for treating Graves' Disease (GD) comprising:
engineering and creating a chimeric autoantigen receptor (CAAR) T cell line for a patient comprising:
selecting a thyroid stimulating hormone receptor (TSHR) epitope predicted to fold autonomously and to bind effectively to a disease-causing, stimulating anti-TSHR antibody (TRAb) of a B cell;
synthesizing a DNA fragment for the TSHR epitope;
cloning the DNA fragment into a CAAR construct as a binding domain, the CAAR construct comprising a vector containing an extracellular binding domain and linker, a transmembrane domain, and a signaling domain;
transfecting the CAAR construct into a lentiviral-generating cell;
collecting and isolating lentivirus for the CAAR construct; and
transducing T cells of the patient with the lentivirus to generate the CARR T cell line;
confirming expression of the CARR construct in the CARR T cell line; and
administering a therapeutic dose of the CARR T cell line expressing the CARR construct to the patient.

2. The method as recited in claim 1, wherein the TSHR epitope comprises a leucine rich repeat (LRR) region where disease-causing, stimulating TRAbs bind.

3. The method as recited in claim 1, wherein:
the transmembrane domain comprises a CD28 transmembrane domain; and
the signaling domain comprises CD28 and CD3ζ signaling domains.

4. The method as recited in claim 1, wherein transfecting the CARR construct into a lentiviral-generating cell comprises co-transfecting the lentiviral-generating cell with the CARR construct and lentiviral packaging and envelope plasmids.

5. The method as recited in claim 4, wherein the lentiviral-generating cell that is transfected is a HEK293FT cell.

6. The method as recited in claim 1, wherein administering the therapeutic dose of the CARR T cell line expressing the CARR construct causes destruction of substantially all autoreactive B cells of the patient expressing TRAbs while not affecting substantially any other B cells of the patient.

7. The method as recited in claim 1, wherein engineering and creating a CAAR T cell line further comprises incorporating a LINK CARR construct in the CAAR T cell line that requires binding of CD19 before the CAAR T cell line is activated, enhancing specificity of the CAAR T cell line to B cells.

8. The method as recited in claim 1, wherein the TSHR epitope comprises an amino acid size of 260 to 290 amino acids (aa).

9. The method as recited in claim 1. wherein the TSHR epitope comprises an amino acid size of 270 to 280 amino acids (aa).

10. A method for treating an autoimmune disease comprising:
engineering and creating a chimeric autoantigen receptor (CAAR) T cell line for a patient comprising:
selecting a receptor epitope predicted to fold autonomously and to bind effectively to a disease-causing antibody (Ab) of a cell of the patient;
synthesizing a DNA fragment for the receptor epitope;
cloning the DNA fragment into a CAAR construct as a binding domain, the CAAR construct comprising a vector containing an extracellular binding domain and linker, a transmembrane domain, and a signaling domain;
transfecting the CAAR construct into a lentiviral-generating cell;
collecting and isolating lentivirus for the CAAR construct; and
transducing T cells of the patient with the lentivirus to generate the CARR T cell line;
confirming expression of the CARR construct in the CARR T cell line; and
administering a therapeutic dose of the CARR T cell line expressing the CARR construct to the patient.

11. The method as recited in claim 10, wherein the epitope comprises a leucine rich repeat (LRR) region where disease-causing, stimulating Abs bind.

12. The method as recited in claim 10, wherein:
the transmembrane domain comprises a CD28 transmembrane domain; and
the signaling domain comprises CD28 and CD3ζ signaling domains.

13. The method as recited in claim 10, wherein transfecting the CARR construct into a lentiviral-generating cell comprises co-transfecting the lentiviral-generating cell with the CARR construct and lentiviral packaging and envelope plasmids.

14. The method as recited in claim 13, wherein the lentiviral-generating cell that is transfected is a HEK293FT cell.

15. The method as recited in claim 10, wherein administering the therapeutic dose of the CARR T cell line expressing the CARR construct causes destruction of substantially all autoreactive disease-causing cells of the patient expressing Abs while not affecting substantially any other non-disease-causing cells of the patient.

16. The method as recited in claim 10, wherein engineering and creating a CAAR T cell line further comprises incorporating a LINK CARR construct in the CAAR T cell line that requires binding of a cell-type-specific receptor before the CAAR T cell line is activated, enhancing specificity of the CAAR T cell line to disease-causing cells.

17. A method for treating Graves' Disease (GD) comprising:
engineering and creating a chimeric autoantigen receptor (CAAR) T cell line for a patient comprising:
selecting a thyroid stimulating hormone receptor (TSHR) epitope comprising a leucine rich repeat (LRR) region predicted to fold autonomously and to bind effectively to a disease-causing, stimulating anti-TSHR antibody (TRAb) of a B cell;
synthesizing a DNA fragment for the TSHR epitope, the DNA fragment comprising:
a TSHR epitope sequence;
a GS linker after the TSHR epitope sequence; and
a BpiI restriction site at each end to facilitate cloning;
cloning the DNA fragment into a CAAR construct as a binding domain, the CAAR construct comprising a vector containing an extracellular binding domain and linker, a CD28 transmembrane domain, and CD28 and CD3ζ signaling domains;
co-transfecting the CAAR construct into a lentiviral-generating cell with lentiviral packaging and envelope plasmids;
collecting and isolating lentivirus for the CAAR construct; and
transducing T cells of the patient with the lentivirus to generate the CARR T cell line; and
administering a therapeutic dose of the CARR T cell line expressing the CARR construct to the patient.

18. The method as recited in claim 17, further comprising confirming expression of the CARR construct in the CARR T cell line.

19. The method as recited in claim 1, wherein engineering and creating a CAAR T cell line further comprises incorporating a LINK CARR construct in the CAAR T cell line that requires binding of CD19 before the CAAR T cell line is activated, enhancing specificity of the CAAR T cell line to B cells.

20. The method as recited in claim 1, wherein the TSHR epitope comprises an amino acid size of 270 to 290 amino acids (aa).
